(19) United States
(12) Patent Application Publication
(10) Pub. No.: US 2019/0323075 A1
(43) Pub. Date: Oct. 24, 2019

(54) Cleavable Competitor Polynucleotides

(71) Applicant: SWIFT BIOSCIENCES, INC., Ann Arbor, MI (US)

(72) Inventor: Vladimir Makarov, Ann Arbor, MI (US)

(21) Appl. No.: 16/503,840

(22) Filed: Jul. 5, 2019

Related U.S. Application Data
(63) Continuation of application No. 15/101,551, filed on Jun. 3, 2016, now Pat. No. 10,385,388.
(60) Provisional application No. 61/912,696, filed on Dec. 6, 2013.

(51) Int. Cl. C12Q 1/6858

(52) U.S. Cl. CPC C12Q 1/6858

(57) ABSTRACT

The invention relates to polynucleotide combinations and their use in allele-specific enrichment, amplification, and detection. The disclosure also provides methods to multiplex various target DNA molecules in a single tube with high sensitivity and specificity.

### CROSS-REFERENCE TO RELATED APPLICATIONS

This application claims the priority benefit under 35 U.S.C. § 119(e) of U.S. Provisional Patent Application No. 61/912,696, filed Dec. 6, 2013, the disclosure of which is incorporated herein by reference in its entirety.

### FIELD OF THE INVENTION

The invention relates to polynucleotide combinations and their use in allele-specific enrichment, amplification and detection.

### INCORPORATION BY REFERENCE OF MATERIAL SUBMITTED ELECTRONICALLY

This application contains, as a separate part of disclosure, a Sequence Listing in computer-readable form (filename: 48198A_SeqListing.txt; created Dec. 5, 2014, 5,854 byte—ASCII text file) which is incorporated by reference in its entirety.

### BACKGROUND

Detection and amplification of nucleic acids play important roles in genetic analysis, molecular diagnostics, and drug discovery. Many such applications require specific, sensitive and cost effective quantitative detection of DNA mutations, copy number variants, gene expression or DNA methylation patterns that are present in a small fraction of total polynucleotides. In the field of cancer diagnosis for instance early detection of somatic mutations greatly increase the survival rate of cancer patients. Monitoring for occurrence of drug resistant mutations is also crucial in determining if a patient will have a relapse of the disease. An ideal example would be EGFR T790M mutation which occurs as a tyrosine kinase inhibitor resistant mutation in several non-small cell lung cancer (NSCLC) patients. Initially NSCLC patients harboring activating mutations in the epidermal growth factor receptor (EGFR) kinase domain tend to respond well to the tyrosine kinase inhibitors, gefitinib and erlotinib. However within a year in most cases relapse occurs due to drug resistance caused by an acquired secondary EGFR kinase domain mutation, T790M. Many of these early somatic mutations and drug resistant mutations are rare mutations which occur within a huge background of non-mutated DNA molecules. Many current methods use polymerase chain reaction (PCR), quantitative PCR (qPCR) and Next Generation Sequencing (NGS) to detect and quantify DNA and RNA variants from clinical samples.

While the performance of qPCR and NGS assays is constantly improving, the sensitivity and specificity of such methods suffer from technical limitations that make the methods inadequate for some applications, such as in detection and discrimination of rare DNA molecules with a single base mutation in situations when they are mixed with thousands of non-mutated DNA molecules. The sensitivity of existing qPCR and NGS technologies are typically limited to 1% and 5% respectively, which is not sufficient for rare allele detection. Another limitation with the current qPCR assays is the ability to combine multiple mutation detection assays into one multiplex diagnostic assay. Multiple mutation detection requires multiple primers and probes which can cause either non-specific amplification of DNA or lead to formation of primer-dimers which greatly reduces the efficiency of a qPCR assay.

There thus remains a need for the development of allelic enrichment and amplification strategies which will enable detection of rare mutations with high sensitivity and specificity. There also remains a need for development for single tube multiplex diagnostic assays which can greatly reduce the cost and time per assay.

### SUMMARY OF THE INVENTION

In one aspect, the disclosure provides a polynucleotide competitor that comprises a sequence that is fully complementary to a first target DNA polynucleotide region (T₁) such that the competitor polynucleotide will hybridize to the first target DNA polynucleotide region under appropriate conditions. In another aspect, the polynucleotide competitor comprises a mismatch to a non-target DNA polynucleotide that is a sequence variant of the first target DNA polynucleotide region (T₁*).

In another aspect, the disclosure provides a polynucleotide competitor that comprises a sequence that is fully complementary to the non-target DNA polynucleotide region (T₁*) such that the competitor polynucleotide will hybridize to the non-target DNA polynucleotide region under appropriate conditions. In another aspect, the polynucleotide competitor comprises a mismatch to a target DNA polynucleotide region T₁ that is a sequence variant of the non-target DNA polynucleotide region.

The disclosure further contemplates an aspect wherein the polynucleotide competitor comprises a single RNA base or a plurality of consecutive RNA bases that are at the position of a mismatched base in the target DNA polynucleotide region (T₁) or the non-target DNA polynucleotide region (T₁*) or alternatively the mismatched base is positioned 1, 2 or 3 DNA bases away either 5′ or 3′ to the RNA base(s). In one aspect, RNA base(s) are located close to the central part of the polynucleotide competitor. In another aspect, RNA base(s) are located within the 3′ portion of the polynucleotide competitor. In still another aspect, the polynucleotide competitor is comprised of RNA bases in its entirety.

In one aspect, a competitor comprising one or more RNA bases enables the polynucleotide competitor to be a substrate for cleavage by RNase H2 upon binding to its target DNA (T₁) and a less efficient substrate for cleavage by RNase H2 upon binding to its non-target DNA (T₁*). In another aspect, a competitor comprising 4 or more consecutive RNA bases enables the polynucleotide competitor to be a substrate for cleavage by RNase H1 upon binding to its target DNA (T₁) and a less efficient substrate for cleavage by RNase H1 upon binding to its non-target DNA (T₁*). In the case of a polynucleotide competitor that is fully complementary to the non-target (T₁*) and which comprises a mismatch to the target polynucleotide (T₁), the cleavage pattern is reversed wherein the polynucleotide competitor is a substrate for cleavage upon binding to the non-target T₁* and is a less efficient substrate for cleavage upon binding to the target T₁ DNA region, utilizing either RNase H1 or RNase H2.

In another aspect, the polynucleotide competitor further comprises a modified nucleic acid, and includes a blocking group to prevent DNA polymerase extension from the 3′ end of the competitor polynucleotide, and optionally includes one or more cleavage-resistant linkages between RNA bases, DNA bases, and DNA-RNA junctions to eliminate cleavage by RNase H enzymes at other potential cleavage sites and to direct RNase H cleavage to the most mismatch sensitive position which is determined empirically for each sequence. Additional cleavage-resistant linkages, in various aspects, are incorporated at the 3′ terminal bases of the competitor polynucleotide to block 3′-5′ exonuclease activity by a proof-reading polymerase.

In another aspect, the polynucleotide competitor further comprises a 5′ sequence that is not complementary to either the target DNA T₁ or to the non-target DNA T₁*.

In another aspect, the disclosure provides a polynucleotide combination comprising a polynucleotide competitor and flanking PCR amplification primers, wherein the polynucleotide competitor comprises a sequence that is fully complementary to a first target DNA polynucleotide region (T₁) such that the competitor polynucleotide will hybridize to its target under appropriate conditions. In another aspect, the polynucleotide competitor comprises a mismatch to a non-target DNA polynucleotide that is a sequence variant of the first target DNA polynucleotide region (T₁*). Alternatively the polynucleotide competitor sequence is fully complementary to the non-target T₁* and harbors a mismatch to the target T₁. In either aspect, the polynucleotide competitor additionally comprises an RNA base, a modified base and a nuclease-resistant linkage as described above to enable the polynucleotide competitor to serve as a substrate for mismatch-sensitive RNase H1 or RNase H2 cleavage under appropriate hybridization and reaction conditions. In combination with the polynucleotide competitor, a PCR amplification primer pair comprises a sequence that is fully complementary to a second target DNA polynucleotide region (T₂) and a third target DNA polynucleotide region (T₃), thus comprising a forward primer where its target sequence T₂ is located 5′ to the competitor target sequence T₁ (and T₁* mismatched non-target) and a reverse primer where its target sequence T₃ is located 3′ to the competitor target sequence T₁ (and T₁* mismatched non-target), where the reverse primer is complementary to the strand that is the reverse complement of the strand which the forward primer and competitor hybridize.

In various aspects of the competitor and primer pair combinations, the forward primer target region T₂ does not overlap with the competitor target region T₁. In other aspects, the forward primer target region T₂ does overlap with the competitor target region T₁, where the area of overlap between T₁ and T₂ is limited to sequences that do not vary between the target DNA T₁ and the non-target DNA T₁* and where the area of overlap is not in sequences that do vary between the target DNA T₁ and the non-target DNA T₁*, where such T₂ and T₃ regions correspond to PCR primers that amplify both T₁ and T₁* equally. In various aspects, the PCR primer pair further comprises a nucleic acid modification, and include, for example and without limitation, nuclease-resistant linkages at 3′ terminal bases to prevent 3′-5′ exonuclease activity by a proof-reading polymerase.

In other aspects of the competitor and primer pair combinations, the forward primer target region T₂ overlaps with the competitor target region T₁, where the area of overlap between T₁ and T₂ includes the sequence variant between the target DNA T₁ and the non-target DNA T₁* and where such a T₂ region corresponds to an allele-specific forward primer that preferentially amplifies the fully complementary T₁ target sequence and either does not amplify T₁* or does so at a reduced efficiency when the mismatch to the T₁* non-target sequence is located at or near the 3′ terminus of the forward primer. In another aspect, the allele-specific primer is comprised of two polynucleotides as previously disclosed in International Application No. PCT/US2010/054362, filed on Oct. 27, 2010, which is incorporated by reference herein in its entirety. The PCR primer pair further comprise nucleic acid modifications, and include nuclease-resistant linkages at 3′ terminal bases to prevent 3′-5′ exonuclease activity by a proof-reading polymerase.

In some aspects, the primer pair target regions T₂ and T₃ correspond to endogenous genomic DNA sequences that are in proximity to an endogenous genomic DNA sequence that corresponds to the competitor target region T₁ and T₁*, where such primers are designated forward and reverse primers. In other aspects, the primer pair target regions T₂ and T₃ correspond to universal DNA library adaptor sequences that are in proximity to an endogenous genomic DNA sequence that corresponds to the competitor target regions T₁ and T₁* when T₁ and T₁* are included in a DNA library (for example, and without limitation, a Next Generation Sequencing or NGS library), where such primers are designated adaptor-specific Primer A and adaptor-specific Primer B.

In each polynucleotide competitor and primer pair combination, an aspect is provided wherein a hairpin detection probe is additionally included, wherein the hairpin portion is comprised of a unique polynucleotide sequence that is not complementary to any target sequence of the disclosure and which forms a hairpin structure at its 5′ end through a self-complementary domain and loop sequence, and where the 3′ domain of the hairpin probe is single-stranded and complementary to the 5′ domain of a competitor polynucleotide of this disclosure, designated C1, and where the hairpin probe additionally includes a nucleic acid modification, including a 5′ terminal fluorophore (or quencher), an internal quencher (or fluorophore) at the junction of the single stranded domain and the self-complementary hairpin structure, and additionally the single stranded domain contains a modified base that increases binding affinity for its complementary sequence C1. Alternatively, if a 5′ domain that is not complementary to the T₁ target but is complementary to the 3′ end of the single stranded domain of the hairpin detection probe is additionally included on the competitor polynucleotide, such a C1 cleavage product would dissociate from the T₁ target at an appropriate reaction temperature and retain the ability to anneal to the single stranded portion of the hairpin detection probe due to the increased length of complementarity.

In an alternate polynucleotide competitor and primer pair combination, an aspect is provided wherein a hairpin detection probe is incorporated on an additional 5′ domain of the competitor polynucleotide, wherein the hairpin domain is comprised of a unique polynucleotide sequence that is not complementary to any target sequence of the disclosure and which forms a hairpin structure at its 5′ end through a self-complementary domain and loop sequence, and where the 3′ domain of the polynucleotide competitor/probe is single-stranded and at its 3′ portion corresponds to the competitor polynucleotide of this disclosure and the 5′ portion of the single-stranded domain corresponds to a C1′ reverse complement of the C1 competitor cleavage product, and where the hairpin probe domain additionally includes a nucleic acid modification, including a 5′ terminal quencher, an internal fluorophore at the junction of the single stranded domain and the self-complementary hairpin structure, and where additionally the single stranded C1′ domain contains modified bases that increase the binding affinity for its complementary sequence C1.

In another polynucleotide competitor and primer pair combination, an aspect is provided wherein a hydrolysis detection probe or a beacon detection probe is additionally included, wherein the hydrolysis detection probe or the beacon detection probe is comprised of a unique polynucleotide sequence complementary to the genomic sequence located between the primer target sequences T₂ and T₃.

In another aspect, the disclosure provides a plurality of polynucleotide combinations, each comprising a polynucleotide competitor and flanking PCR primers, where a first competitor comprises a sequence that is fully complementary to a first target DNA polynucleotide region (T₁) and comprises a mismatch to a non-target DNA polynucleotide that is a sequence variant of the first target DNA polynucleotide region (T₁*), and the first PCR primer pair comprises sequences that are fully complementary to a second target DNA polynucleotide region (T₂) and a third target DNA polynucleotide region (T₃), thus comprising a forward primer where its target sequence T₂ is located 5′ to the competitor target sequence T₁ (where T₂ and T₁ are either distinct or partially overlapping), and a reverse primer where its target sequence T₃ is located 3′ to the competitor target sequence T₁, where the 3′ reverse primer is complementary to the strand that is the reverse complement of the strand which the forward primer and competitor hybridize. A second competitor comprises a sequence that is fully complementary to a fourth target DNA polynucleotide region (T₄) and comprises a mismatch to a non-target DNA polynucleotide that is a sequence variant of the fourth target DNA polynucleotide region (T₄*), and a second PCR amplification primer pair comprises sequences that are fully complementary to a fifth target DNA polynucleotide region (T₅) and a sixth target DNA polynucleotide region (T₆) that flank T₄ and T₄*, and in an ‘n’ plurality of polynucleotide combinations where ‘n’ competitors comprise sequences that are fully complementary to ‘n’ target DNA polynucleotide regions (n₁) and comprise a mismatch to non-target DNA polynucleotides that are sequence variants (n₁*), and ‘n’ PCR primer pairs comprise sequences that are fully complementary to (n₂) and (n₃) target DNA polynucleotide regions that flank n₁ and n₁*. In another aspect, an ‘n’ plurality of polynucleotide competitors are paired with NGS adaptor-specific Primers A and B when ‘n’ competitor target regions are incorporated into a DNA(NGS) library.

Also contemplated in this disclosure is a method of cleaving a polynucleotide competitor that comprises a sequence that is fully complementary to a first target DNA polynucleotide region (T₁) such that the competitor polynucleotide will hybridize to its target under appropriate conditions. The method comprises (i) contacting a first target polynucleotide with a polynucleotide competitor under conditions wherein the polynucleotide competitor hybridizes to the first target polynucleotide sequence to form a first competitor/target complex; (ii) contacting the competitor/target complex with an enzyme that specifically identifies and cleaves the competitor/target complex when it is a fully complementary complex; and (iii) optionally detecting the enzyme cleavage. In another aspect, the polynucleotide competitor comprises a mismatch to a non-target DNA polynucleotide that is a sequence variant of the first target DNA polynucleotide region (T₁*). The disclosure further contemplates an aspect wherein the polynucleotide competitor comprises an RNA base that is at the position of the mismatched base to the non-target DNA region (T₁*), or alternatively the mutation lies 1, 2 or 3 bases away either 5′ or 3′ to the RNA base. In one aspect, the RNA base enables the polynucleotide competitor to be a substrate for cleavage by RNase H2 upon binding to its target DNA (T₁) and a less efficient substrate for cleavage by RNase H2 upon binding to its non-target DNA (T₁*), where under appropriate reaction conditions known in the art, the addition of RNase H2 enzyme will lead to cleavage of the competitor polynucleotide at the position of the RNA base when it is annealed to its fully complementary target sequence T₁ and not when it remains unbound in single-stranded form or if it is annealed to a mismatched non-target sequence T₁* or other mismatched non-target sequence. In another aspect, there is more than one RNA base within the competitor polynucleotide that is a substrate for cleavage by RNase H2 upon binding to its target DNA (T₁) and a less efficient substrate for cleavage by RNase H2 upon binding to its non-target DNA (T₁*).

The disclosure further provides a method of cleaving a polynucleotide competitor that comprises a sequence that is fully complementary to a first target DNA polynucleotide region (T₁) such that the competitor polynucleotide will hybridize to its target under appropriate conditions. The method comprises (i) contacting a first target polynucleotide with a polynucleotide competitor under conditions wherein the polynucleotide competitor hybridizes to the first target polynucleotide sequence to form a first competitor/target complex; (ii) contacting the competitor/target complex with an enzyme that specifically identifies and cleaves the competitor/target complex when it is a fully complementary complex; and (iii) optionally detecting the enzyme cleavage. In another aspect, the polynucleotide competitor comprises a mismatch to a non-target DNA polynucleotide that is a sequence variant of the first target DNA polynucleotide region (T₁*). The disclosure further contemplates an aspect wherein the polynucleotide competitor has a domain comprising 4 or more consecutive RNA bases, one of which is at the position of the mismatched base to the non-target DNA region (T₁*) or alternatively the mismatch lies 1, 2 or 3 DNA bases away either 5′ or 3′ to the RNA domain. In one aspect, the domain comprising 4 or more RNA bases enables the polynucleotide competitor to be a substrate for cleavage by RNase H1 or RNase H2 upon binding to its target DNA (T₁) and a less efficient substrate for cleavage by RNase H1 or RNase H2 upon binding to its non-target DNA (T₁*), where under appropriate reaction conditions known in the art, the addition of RNase H1 or RNase H2 enzyme will lead to cleavage of the competitor polynucleotide at a position within the 4 or more consecutive RNA bases when it is annealed to its fully complementary target sequence T₁ and not when it remains unbound in single-stranded form or if it is annealed to a mismatched non-target sequence T₁* or other mismatched non-target sequence.

In another aspect, the disclosure provides a method of allelic enrichment by amplification utilizing a polynucleotide combination comprising a cleavable competitor and flanking non-allele-specific PCR primers, where the cleavable competitor comprises a sequence that is fully complementary to a first target DNA polynucleotide region (T₁) such that the competitor polynucleotide will hybridize to its target under appropriate conditions. The method comprises (i) contacting a first target polynucleotide with a polynucleotide competitor under conditions wherein the polynucleotide competitor hybridizes to the first target polynucleotide sequence to form a first competitor/target complex; (ii) contacting the competitor/target complex with an enzyme that specifically identifies and cleaves the competitor/target complex when it is a fully complementary complex, wherein competitor cleavage leads to cleavage product dissociation and enables a DNA polymerase to extend the forward primer and amplification of T₁ occurs via PCR; and (iii) optionally detecting the enzyme cleavage. In another aspect, the polynucleotide competitor comprises a mismatch to a non-target DNA polynucleotide that is a sequence variant of the first target DNA polynucleotide region (T₁*). The disclosure further contemplates an aspect wherein the polynucleotide competitor comprises an RNA domain that is at the position of the mismatched base to the non-target DNA region (T₁*), or alternatively the mutation lies 1, 2 or 3 bases away either 5′ or 3′ to the RNA domain. In one aspect, the RNA domain enables the polynucleotide competitor to be a substrate for cleavage by RNase H1 or RNase H2 upon binding to its target DNA (T₁) and a less efficient substrate for cleavage upon binding to its non-target DNA (T₁*). Also included in this method is a PCR primer pair, where the primer pair comprises sequences that are fully complementary to second (T₂) and third (T₃) target DNA polynucleotide regions, thus comprising a forward primer where its target sequence T₂ is located 5′ to the competitor target sequence T₁ (where T₂ and T₁ are either distinct or partially overlapping but where T₂ does not include the variant base between T₁ and T₁*), and a reverse primer where its target sequence T₃ is located 3′ to the competitor target sequence T₁, where the 3′ reverse primer is complementary to the strand that is the reverse complement of the strand which the forward primer and competitor hybridize, and where T₂ and T₃ target DNA polynucleotide regions do not vary in sequence relative to the target DNA polynucleotide T₁ and the non-target DNA polynucleotide T₁*, where under appropriate reaction conditions known in the art, the addition of RNase H1 or RNase H2 enzyme will lead to cleavage of the competitor polynucleotide at a position within the RNA domain or at the DNA-RNA junction when it is annealed to its fully complementary target sequence T₁ and not when it remains unbound in single-stranded form or if it is annealed to a mismatched non-target sequence T₁* or other mismatched non-target sequence, and in the same reaction a DNA polymerase, with the inclusion of other reagents required for PCR amplification, the target sequence T₁ will be selectively amplified over the non-target sequence T₁*, whereby competitor cleavage on the T₁ strand will lead to cleavage product dissociation and enable a DNA polymerase to extend the forward primer and T₁ PCR amplification can occur, whereby on the non-target T₁*, the intact competitor will remain annealed and prevent a DNA polymerase from extending the forward primer on the non-target T₁* template and T₁* PCR amplification will be suppressed.

In an aspect of the embodiment wherein the forward primer overlaps with the competitor sequence, the RNase H1 or RNase H2 cleavage and simultaneous PCR amplification are performed at two reaction temperatures following denaturation, the first of which enables annealing of the intact, uncleaved competitor but which reaction temperature exceeds the annealing temperature of the forward and reverse primers and competitor cleavage products C1 and C2 which subsequently dissociate from their template following cleavage by RNase H1 or RNase H2, whereby the second, lower temperature that still exceeds the annealing temperature of the competitor cleavage products C1 and C2 but enables the forward and reverse primers to bind the target T₁ template and allele-enriched amplification can proceed, whereby on the non-target T₁* strand the uncleaved competitor prevents the forward primer from annealing.

In an aspect of the embodiment when the forward primer does not overlap with the competitor sequence, the RNase H1 or RNase H2 cleavage and simultaneous PCR amplification are also performed at two reaction temperatures following denaturation, the first of which enables annealing of the intact, uncleaved competitor but which reaction temperature exceeds the annealing temperature of the forward and reverse primers and competitor cleavage products C1 and C2 which subsequently dissociate from their template following cleavage by RNase H1 or RNase H2, whereby the second, lower temperature that still exceeds the annealing temperature of the competitor cleavage products C1 and C2 but enables the forward and reverse primers to bind the target T₁ and T₁* templates and specifically enables forward primer extension on the T₁ template whereby on the T₁* template forward primer extension is blocked by the uncleaved competitor.

In each method utilizing polynucleotide combinations of this disclosure for allele-enriched cleavable competitor PCR (or allele-enriched PCR with cleavable competitor), an aspect to the method is provided wherein a hairpin detection probe is additionally included, wherein the hairpin portion is comprised of a unique polynucleotide sequence that is not complementary to any target sequence of the disclosure and which forms a hairpin structure at its 5′ end through a self-complementary domain and loop sequence, and where the 3′ domain of the hairpin probe is single-stranded and complementary to the 5′ domain of a competitor polynucleotide of this disclosure, designated C1, and where the hairpin probe additionally includes nucleic acid modifications, including a 5′ terminal fluorophore (or quencher), an internal quencher (or fluorophore) at the junction of the single stranded domain and the self-complementary hairpin structure, and additionally the single stranded domain may contain modified bases or additional sequence complementary to the C1 cleavage product that increase the binding affinity for its complementary sequence C1, where under appropriate reaction conditions, upon allele-specific cleavage of the competitor polynucleotide with RNase H1 or RNase H2, annealing and extension of the C1 competitor cleavage product on the hairpin detection probe unfolds the self-complementary portion of the hairpin detection probe, thus physically separating the fluorophore from the quencher which were previously juxtaposed and where the proximity results in quenching of the fluorophore signal, and where physical separation produces a fluorescence signal, and where fluorescence detection indicates the presence of the target sequence T₁ and which fluorescence signal is increased proportionally during PCR amplification of the target sequence T₁. In another aspect, the hairpin detection probe can be incorporated into the 5′ domain of the competitor, where upon competitor cleavage, the C1 cleavage portion can anneal to its C1′ complement and polymerase extension of the C1 cleavage product on the hairpin detection probe unfolds the self-complementary portion of the probe, thus physically separating the fluorophore from the quencher which were previously juxtaposed and where the proximity results in quenching of the fluorophore signal, and where physical separation produces a fluorescence signal, and where fluorescence detection indicates the presence of the target sequence T₁ and which fluorescence signal is increased proportionally during PCR amplification of the target sequence T₁.

In an alternative embodiment, the allele-enriched cleavable competitor PCR (or allele-enriched PCR with cleavable competitor) does not involve a simultaneous detection step, where a subsequent allele-specific qPCR is performed to detect the presence of target sequence T₁, or, the allele-enriched competitor PCR is performed on an NGS library or an NGS library is simultaneously or subsequently made from the product of the allele-enriched competitor PCR and detection of target sequence T₁ is performed by NGS analysis.

In another aspect, the disclosure provides a multiplexed method of allele-enriched cleavable competitor PCR (or allele-enriched PCR with cleavable competitor) using a plurality of polynucleotide combinations, each comprising a cleavable competitor and flanking non-allele-specific PCR primers. The method comprises (i) contacting each target polynucleotide of the ‘n’ plurality with ‘n’ polynucleotide competitors under conditions wherein each polynucleotide competitor hybridizes to its corresponding target polynucleotide sequence to form a competitor/target complex; (ii) contacting ‘n’ competitor/target complexes with an enzyme that specifically identifies and cleaves the competitor/target complexes when they are fully complementary complexes, wherein competitor cleavage leads to cleavage product dissociation and enables a DNA polymerase to extend ‘n’ forward primers and PCR amplification of ‘n’ target polynucleotides occurs; and (iii) optionally detecting the enzyme cleavage. A first competitor comprises a sequence that is fully complementary to a first target DNA polynucleotide region (T₁) and comprises a mismatch to a non-target DNA polynucleotide that is a sequence variant of the first target DNA polynucleotide region (T₁*), and the first PCR amplification primer pair comprises sequences that are fully complementary to second (T₂) and third (T₃) target DNA polynucleotide regions, thus comprising a forward primer where its target sequence T₂ is located 5′ to the competitor target sequence T₁ (where T₂ and T₁ are either distinct or partially overlapping but where T₂ does not include the variant base between T₁ and T₁*), and a reverse primer where its target sequence T₃ is located 3′ to the competitor target sequence T₁, where the 3′ reverse primer is complementary to the strand that is the reverse complement of the strand to which the forward primer and competitor hybridize. A second comprises a sequence that is fully complementary to a fourth target DNA polynucleotide region (T₄) and comprises a mismatch to a non-target DNA polynucleotide that is a sequence variant of the fourth target DNA polynucleotide region (T₄*), and a second PCR amplification primer pair comprises sequences that are fully complementary to fifth (T₅) and sixth (T₆) target DNA polynucleotide regions that flank T₄ and T₄*, and in an ‘n’ plurality of polynucleotide combinations where ‘n’ competitors comprise sequences that are fully complementary to ‘n’ target DNA polynucleotide regions (n₁) and comprise a mismatch to non-target DNA polynucleotides that are sequence variants (n₁*), and ‘n’ PCR primer pairs comprise sequences that are fully complementary to (n₂) and (n₃) target DNA polynucleotide regions that flank n₁ and n₁*, where under appropriate reaction conditions known in the art, the addition of RNase H1 or RNase H2 enzyme will lead to cleavage of ‘n’ competitor polynucleotides at a position within the RNA bases or at the DNA-RNA junction when they are annealed to their fully complementary target sequence ‘n₁’ and not when they remain unbound in single-stranded form or if they are annealed to a mismatched non-target sequence ‘n₁*’ or other mismatched non-target sequence, and in the same reaction a DNA polymerase, with the inclusion of other reagents required for PCR amplification, the target sequences ‘n₁’ will be selectively amplified over the non-target sequences ‘n₁*’, where competitor cleavage on each ‘n₁’ strand will lead to competitor cleavage product dissociation and enable a DNA polymerase to extend the forward primer and ‘n₁’ PCR amplification can occur, where on each non-target ‘n₁*’strand, the intact competitor polynucleotides will remain annealed and prevent a DNA polymerase from extending the forward primer on the non-target ‘n₁*’ template and ‘n₁*’ PCR amplification will be suppressed.

In an aspect of the embodiment when the forward primer overlaps with the competitor sequence, the RNase H1 or RNase H2 cleavage and simultaneous PCR amplification are performed at two reaction temperatures following denaturation, the first of which enables annealing of the intact, uncleaved competitor but which reaction temperature exceeds the annealing temperature of the forward and reverse primers and competitor cleavage products C1 and C2 which subsequently dissociate from their template following cleavage by RNase H1 or RNase H2, whereby the second, lower temperature that still exceeds the annealing temperature of the competitor cleavage products C1 and C2 but enables the forward and reverse primers to bind the target T₁ template and allele-enriched amplification can proceed, whereby on the non-target T₁* strand the uncleaved competitor prevents the forward primer from annealing.

In an aspect of the embodiment when the forward primer does not overlap with the competitor sequence, the RNase H1 or RNase H2 cleavage and simultaneous PCR amplification are also performed at two reaction temperatures following denaturation, the first of which enables annealing of the intact, uncleaved competitor but which reaction temperature exceeds the annealing temperature of the forward and reverse primers and competitor cleavage products C1 and C2 which subsequently dissociate from their template following cleavage by RNase H1 or RNase H2, whereby the second, lower temperature that still exceeds the annealing temperature of the competitor cleavage products C1 and C2 but enables the forward and reverse primers to bind the target T₁ and T₁* templates and specifically enables forward primer extension on the T₁ template whereby on the T₁* template forward primer extension is blocked by the uncleaved competitor.

In each method utilizing polynucleotide combinations of this disclosure for multiplexed allelic-enriched competitor PCR, an aspect of the method is provided wherein a plurality of ‘n’ hairpin detection probes are additionally included for multiplexed detection, wherein each hairpin detection probe of the plurality utilizes a unique fluorophore that can be distinguished during the detection step of the reaction, where under appropriate reaction conditions, upon allele-specific cleavage of ‘n’ competitor polynucleotides, annealing and extension of ‘n’ C1 competitor cleavage products on ‘n’ hairpin detection probes unfolds the self-complementary portion of ‘n’ hairpin detection probes, thus physically separating ‘n’ fluorophores from their quenchers, and where physical separation produces a fluorescence signal, and where multiplexed fluorescence detection indicates the presence of target sequences ‘n₁’ and which fluorescence signals are increased proportionally during PCR amplification of target sequences ‘n₁’. In another aspect, ‘n’ hairpin detection probes can be incorporated into a 5′ domain of corresponding ‘n’ competitor polynucleotides as described above.

In an alternative embodiment, the multiplexed allele-enriched cleavable competitor PCR (or allele-enriched PCR with cleavable competitor) does not involve a simultaneous detection step, where a subsequent single or multiplexed allele-specific qPCR is performed to detect the presence of target sequences ‘n₁’, or, the multiplexed allele-enriched competitor PCR is performed on an NGS library or an NGS library is simultaneously or subsequently made from the product of the multiplexed allele-enriched competitor PCR and detection of target sequences ‘n₁’ is performed by NGS analysis.

In another aspect, the disclosure provides a method of allele-specific amplification utilizing a polynucleotide combination comprising a cleavable competitor and flanking allele-specific PCR primers, where the cleavable competitor comprises a sequence that is fully complementary to a first target DNA polynucleotide region (T₁) such that the competitor polynucleotide will hybridize to its target under appropriate conditions. The method comprises (i) contacting a first target polynucleotide with a polynucleotide competitor under conditions wherein the polynucleotide competitor hybridizes to the first target polynucleotide sequence to form a first competitor/target complex; (ii) contacting the competitor/target complex with an enzyme that specifically identifies and cleaves the competitor/target complex when it is a fully complementary complex, wherein competitor cleavage on the T₁ strand will lead to cleavage product dissociation and enable a DNA polymerase to extend the forward primer and amplification of T₁ occurs via PCR; and (iii) optionally detecting the enzyme cleavage . In another aspect, the polynucleotide competitor comprises a mismatch to a non-target DNA polynucleotide that is a sequence variant of the first target DNA polynucleotide region (T₁*). The disclosure further contemplates an aspect wherein the polynucleotide competitor comprises an RNA domain that is at the position of the mismatched base to the non-target DNA region (T₁*), or alternatively the mutation lies 1, 2 or 3 bases away either 5′ or 3′ to the RNA domain. In one aspect, the RNA domain enables the polynucleotide competitor to be a substrate for cleavage by RNase H1 or RNase H2 upon binding to its target DNA (T₁) and a less efficient substrate for cleavage upon binding to its non-target DNA (T₁*). Also included in this method is an allele-specific PCR primer pair, where the primer pair comprises sequences that are fully complementary to second (T₂) and third (T₃) target DNA polynucleotide regions, thus comprising a forward primer where its target sequence T₂ is located 5′ to the competitor target sequence T₁ and overlaps with T₁ to include the variant base at the 3′ terminus of the forward primer (which results in an allele-specific primer), and a reverse primer where its target sequence T₃ is located 3′ to the competitor target sequence T₁, where the 3′ reverse primer is complementary to the strand that is the reverse complement of the strand which the forward primer and competitor hybridize, and where the T₂ target DNA polynucleotide region varies in sequence relative to the target DNA polynucleotide T₁ and the non-target DNA polynucleotide T₁*, where under appropriate reaction conditions known in the art, the addition of RNase H1 or RNase H2 enzyme will lead to cleavage of the competitor polynucleotide at a position within the RNA domain or at the DNA-RNA junction when it is annealed to its fully complementary target sequence T₁ and not when it remains unbound in single-stranded form or if it is annealed to a mismatched non-target sequence T₁* or other mismatched non-target sequence, and in the same reaction a DNA polymerase, with the inclusion of other reagents required for PCR amplification, the target sequence T₁ will be selectively amplified over the non-target sequence T₁*, whereby competitor cleavage on the T₁ strand will lead to cleavage product dissociation and enable a DNA polymerase to extend the fully complementary allele-specific forward primer and T₁ PCR amplification can occur, whereby on the non-target T₁*, the intact competitor will remain annealed and prevent a DNA polymerase from extending the mismatched allele-specific forward primer on the non-target T₁* template and T₁* PCR amplification will be suppressed.

In an aspect of the embodiment, the RNase H1 or RNase H2 cleavage and simultaneous PCR amplification are performed at two reaction temperatures following denaturation, the first of which enables annealing of the intact, uncleaved competitor but which reaction temperature exceeds the annealing temperature of the forward and reverse primers and competitor cleavage products C1 and C2 which subsequently dissociate from their template following cleavage by RNase H1 or RNase H2, whereby the second, lower temperature that still exceeds the annealing temperature of the competitor cleavage products C1 and C2 but enables the forward and reverse primers to bind the target T₁ template and allele-enriched amplification can proceed, whereby on the non-target T₁* strand the uncleaved competitor prevents the forward primer from annealing.

In each method utilizing polynucleotide combinations of this disclosure for allele-specific PCR, an aspect to the method is provided wherein a hairpin detection probe is additionally included, wherein the hairpin portion is comprised of a unique polynucleotide sequence that is not complementary to any target sequence of the disclosure and which forms a hairpin structure at its 5′ end through a self-complementary domain and loop sequence, and where the 3′ domain of the hairpin probe is single-stranded and complementary to the 5′ domain of a competitor polynucleotide of this disclosure, designated C1, and where the hairpin probe additionally includes nucleic acid modifications, including a 3′ terminal fluorophore (or quencher), an internal quencher (or fluorophore) at the junction of the single stranded domain and the self-complementary hairpin structure, and additionally the single stranded domain contains modified bases or additional 5′ sequence that increases the binding affinity for its complementary sequence C1, where under appropriate reaction conditions, upon allele-specific cleavage of the competitor polynucleotide with RNase H1 or RNase H2, annealing and extension of the C1 competitor cleavage product on the hairpin detection probe unfolds the self-complementary portion of the hairpin detection probe, thus physically separating the fluorophore from the quencher which were previously juxtaposed and where the proximity results in quenching of the fluorophore signal, and where physical separation produces a fluorescence signal, and where fluorescence detection indicates the presence of the target sequence T₁ and which fluorescence signal is increased proportionally during PCR amplification of the target sequence T₁. In another aspect, the hairpin detection probe can be incorporated into the 5′ domain of the competitor, where upon competitor cleavage, the C1 cleavage portion can anneal to its C1′ complement and polymerase extension of the C1 cleavage product on the hairpin detection probe unfolds the self-complementary portion of the probe, thus physically separating the fluorophore from the quencher which were previously juxtaposed and where the proximity results in quenching of the fluorophore signal, and where physical separation produces a fluorescence signal, and where fluorescence detection indicates the presence of the target sequence T₁ and which fluorescence signal is increased proportionally during PCR amplification of the target sequence T₁.

In another aspect, the disclosure provides a multiplexed method of allele-specific competitor PCR using a plurality of polynucleotide combinations, each comprising a cleavable competitor and flanking allele-specific PCR primers. The method comprises (i) contacting each target polynucleotide of the ‘n’ plurality with ‘n’ polynucleotide competitors under conditions wherein each polynucleotide competitor hybridizes to its corresponding target polynucleotide sequence to form a competitor/target complex; (ii) contacting ‘n’ competitor/target complexes with an enzyme that specifically identifies and cleaves the competitor/target complexes when they are fully complementary complexes, wherein competitor cleavage leads to cleavage product dissociation and enables a DNA polymerase to extend ‘n’ forward primers and PCR amplification of ‘n’ target polynucleotides occurs; and (iii) optionally detecting the enzyme cleavage. A first competitor comprises a sequence that is fully complementary to a first target DNA polynucleotide region (T₁) and comprises a mismatch to a non-target DNA polynucleotide that is a sequence variant of the first target DNA polynucleotide region (T₁*), and the first allele-specific PCR amplification primer pair comprises sequences that are fully complementary to second (T₂) and third (T₃) target DNA polynucleotide regions, thus comprising a forward primer where its target sequence T₂ is located 5′ to the competitor target sequence T₁ and overlaps with the variant base such that the forward primer has the variant base at its 3′ terminus, and a reverse primer where its target sequence T₃ is located 3′ to the competitor target sequence T₁, where the 3′ reverse primer is complementary to the strand that is the reverse complement of the strand which the forward primer and competitor hybridize. A second polynucleotide competitor comprises a sequence that is fully complementary to a fourth target DNA polynucleotide region (T₄) and comprises a mismatch to a non-target DNA polynucleotide that is a sequence variant of the fourth target DNA polynucleotide region (T₄*), and a second allele-specific PCR primer pair comprises sequences that are fully complementary to fifth (T₅) and sixth (T₆) target DNA polynucleotide regions that flank T₄ and T₄*, where the forward primer contains the variant base at its 3′ terminus, and in an ‘n’ plurality of polynucleotide combinations where ‘n’ competitors comprise sequences that are fully complementary to ‘n’ target DNA polynucleotide regions (n₁) and comprise a mismatch to non-target DNA polynucleotides that are sequence variants (n₁*), and ‘n’ allele-specific PCR primer pairs comprise sequences that are fully complementary to (n₂) and (n₃) target DNA polynucleotide regions that flank n₁ and n₁*, where each forward primer is allele-specific and contains the variant base at its 3′ terminus, and where under appropriate reaction conditions known in the art, the addition of RNase H1 or RNase H2 enzyme will lead to cleavage of ‘n’ competitor polynucleotides at a position within the RNA bases or at the DNA-RNA junction when they are annealed to their fully complementary target sequence ‘n₁’ and not when they remain unbound in single-stranded form or if they are annealed to a mismatched non-target sequence ‘n₁*’ or other mismatched non-target sequence, and in the same reaction a DNA polymerase , with the inclusion of other reagents required for PCR amplification, the target sequences ‘n₁’ will be selectively amplified over the non-target sequences ‘n₁*’, where competitor cleavage on each ‘n₁’ strand will lead to competitor cleavage product dissociation and enable a DNA polymerase to extend the fully complementary allele-specific forward primer and ‘n₁’ PCR amplification can occur, where on each non-target ‘n₁*’strand, the intact competitor polynucleotides will remain annealed and prevent a DNA polymerase from extending the mismatched forward primer on the non-target ‘n₁*’ template and ‘n₁*’ PCR amplification will be suppressed.

In an aspect of the embodiment, the RNase H1 or RNase H2 cleavage and simultaneous PCR amplification are performed at two reaction temperatures following denaturation, the first of which enables annealing of the intact, uncleaved competitor but which reaction temperature exceeds the annealing temperature of the forward and reverse primers and competitor cleavage products C1 and C2 which subsequently dissociate from their template following cleavage by RNase H1 or RNase H2, whereby the second, lower temperature that still exceeds the annealing temperature of the competitor cleavage products C1 and C2 but enables the forward and reverse primers to bind the target T₁ template and allele-enriched amplification can proceed, whereby on the non-target T₁* strand the uncleaved competitor prevents the forward primer from annealing.

In each method utilizing polynucleotide combinations of this disclosure for multiplexed allele-specific competitor PCR, an aspect to the method is provided wherein a plurality of ‘n’ hairpin detection probes are additionally included for multiplexed detection, wherein each hairpin detection probe of the plurality utilizes a unique fluorophore that can be distinguished during the detection step of the reaction, where under appropriate reaction conditions, upon allele-specific cleavage of ‘n’ competitor polynucleotides, annealing and extension of ‘n’ C1 competitor cleavage products on ‘n’ hairpin detection probes unfolds the self-complementary portion of ‘n’ hairpin detection probes, thus physically separating ‘n’ fluorophores from their quenchers, and where physical separation produces a fluorescence signal, and where multiplexed fluorescence detection indicates the presence of target sequences ‘n₁’ and which fluorescence signals are increased proportionally during PCR amplification of target sequences ‘n₁’. In another aspect, ‘n’ hairpin detection probes can be incorporated into a 5′ domain of corresponding ‘n’ competitor polynucleotides as described above.

In another aspect, the disclosure provides a method of allelic enrichment amplification utilizing a polynucleotide combination comprising an extendable cleavable competitor and flanking PCR primers, where the extendable cleavable competitor comprises a sequence that is fully complementary to a non-target DNA polynucleotide region (T₁*) such that the competitor polynucleotide will hybridize to the non-target under appropriate conditions. The method comprises (i) contacting a first non-target polynucleotide with a polynucleotide competitor under conditions wherein the polynucleotide competitor hybridizes to the first non-target polynucleotide sequence to form a first competitor/non-target complex; (ii) contacting the competitor/non-target complex with an enzyme that specifically identifies and cleaves the competitor/non-target complex when it is a fully complementary complex, wherein competitor cleavage on the non-target strand will lead to 3′ cleavage product dissociation and enable a DNA polymerase to extend the 5′ cleavage product; and wherein when the reaction temperature is raised above the non-cleaved and non-extended cleavable competitor but remains below that of the cleaved and extended competitor, the non-cleaved and non-extended competitor dissociates from the target DNA strand enabling a forward primer to be extended by a DNA polymerase and target-specific PCR amplification occurs; and (iii) optionally detecting the enzyme cleavage. In another aspect, the polynucleotide competitor comprises a mismatch to a target DNA polynucleotide that is a sequence variant of the first non-target DNA polynucleotide region (T₁). The disclosure further contemplates an aspect wherein the extendable cleavable competitor comprises an RNA domain that is at the position of the mismatched base to the target DNA region (T₁), or alternatively the mutation lies 1, 2 or 3 bases away either 5′ or 3′ to the RNA domain. In one aspect, the RNA domain enables the polynucleotide competitor to be a substrate for cleavage by RNase H1 or RNase H2 upon binding to the fully complementary non-target DNA (T₁*) and a less efficient substrate for cleavage upon binding to the mismatched target DNA (T₁), and where the RNA domain is placed such that the 5′ cleavage product C1 remains bound following cleavage and the 3′ cleavage product C2 dissociates. Also included in this method is a PCR primer pair, where the primer pair comprises sequences that are fully complementary to second (T₂) and third (T₃) target DNA polynucleotide regions, thus comprising a forward primer where its target sequence T₂ is located 5′ to the extendable competitor target sequence T₁* and a reverse primer where its target sequence T₃ is located 3′ to the competitor target sequence T₁*, where the 3′ reverse primer is complementary to the strand that is the reverse complement of the strand which the forward primer and competitor hybridize, and where T₂ and T₃ target DNA polynucleotide regions do not vary in sequence relative to the target DNA polynucleotide T₁ and the non-target DNA polynucleotide T₁*, where under appropriate reaction conditions known in the art, the addition of RNase H1 or RNase H2 enzyme will lead to cleavage of the competitor polynucleotide at a position within the RNA domain or at the DNA-RNA junction when it is annealed to the fully complementary non-target sequence T₁* and not when it remains unbound in single-stranded form or if it is annealed to a mismatched target sequence T₁ or other mismatched non-target sequence, and in the same reaction a DNA polymerase , with the inclusion of other reagents required for PCR amplification, the target sequence T₁ will be selectively amplified over the non-target sequence T₁*, whereby competitor cleavage on the T₁* strand will lead to 3′ cleavage product dissociation and enable the 5′ cleavage product to be extended by DNA polymerase, thereby increasing the melting temperature of the cleavage product above that of the uncleaved competitor polynucleotide, which thereby at a temperature above that of the intact competitor Tm and below that of the extended competitor Tm, enables a DNA polymerase to fully extend the already partially extended forward primer and T₁ PCR amplification can occur, whereby on the non-target T₁*, the extended competitor will remain annealed and prevent a DNA polymerase from extending the forward primer on the non-target T₁* template and T₁* PCR amplification will be suppressed.

In an aspect of the embodiment, the RNase H1 or RNase H2 cleavage and simultaneous PCR amplification are performed at multiple reaction temperatures following denaturation, the first of which enables annealing of the intact, uncleaved competitor at temperature t₁ and is followed by annealing and partial extension of the forward primer, annealing and extension of the reverse primer and at the same time competitor cleavage and extension on the T₁* non-target strand at temperature t₂, then when the temperature t₃ exceeds the annealing temperature of the intact competitor which subsequently dissociates from the T₁ target template, allowing the forward primer to complete extension on the T₁ target strand but not the T₁* non-target strand due to the stabilized extended competitor.

In an alternative embodiment, the allelic enriched extendable competitor PCR does not involve a simultaneous detection step, where a subsequent allele-specific qPCR is performed to detect the presence of target sequence T₁, or, the allelic enriched extendable competitor PCR is performed on an NGS library or an NGS library is simultaneously or subsequently made from the product of the allelic enriched extendable competitor PCR and detection of target sequence T₁ is performed by NGS analysis.

In another aspect, the disclosure provides a multiplexed method of allelic enriched extendable competitor PCR using a plurality of polynucleotide combinations, each comprising an extendable cleavable competitor and flanking PCR primers. The method comprises (i) contacting ‘n’ non-target polynucleotides with polynucleotide competitors under conditions wherein the polynucleotide competitors hybridize to their corresponding non-target polynucleotide sequences to form ‘n’ competitor/non-target complexes; (ii) contacting the competitor/non-target complexes with an enzyme that specifically identifies and cleaves the competitor/non-target complexes when they are fully complementary complexes, wherein competitor cleavage of ‘n’ non-target strands will lead to 3′ cleavage product dissociation and enable a DNA polymerase to extend ‘n’ 5′ cleavage products, and wherein when the reaction temperature is raised above the non-cleaved and non-extended cleavable competitors but remains below that of the cleaved and extended competitors, the non-cleaved and non-extended competitors dissociate from ‘n’ target DNA strands enabling ‘n’ forward primers to be extended by a DNA polymerase and ‘n’ target-specific PCR amplification occurs; and (iii) optionally detecting the enzyme cleavage. A first competitor comprises a sequence that is fully complementary to a first non-target DNA polynucleotide region (T₁*) and comprises a mismatch to a target DNA polynucleotide that is a sequence variant of the first target DNA polynucleotide region (T₁), and the first PCR amplification primer pair comprises sequences that are fully complementary to second (T₂) and third (T₃) target DNA polynucleotide regions, thus comprising a forward primer where its target sequence T₂ is located 5′ to the competitor target sequence T₁ and a reverse primer where its target sequence T₃ is located 3′ to the competitor target sequence T₁, where the 3′ reverse primer is complementary to the strand that is the reverse complement of the strand which the forward primer and competitor hybridize. A second polynucleotide combination comprises a sequence that is fully complementary to a second non-target DNA polynucleotide region (T₄*) and comprises a mismatch to a target DNA polynucleotide that is a sequence variant of the fourth target DNA polynucleotide region (T₄), and a second PCR amplification primer pair comprises sequences that are fully complementary to fifth (T₅) and sixth (T₆) target DNA polynucleotide regions that flank T₄ and T₄*, and in an ‘n’ plurality of polynucleotide combinations where ‘n’ competitors comprise sequences that are fully complementary to ‘n’ non-target DNA polynucleotide regions (n₁*) and comprise a mismatch to target DNA polynucleotides that are sequence variants (n₁), and ‘n’ PCR primer pairs comprise sequences that are fully complementary to (n₂) and (n₃) target DNA polynucleotide regions that flank n₁ and n₁*, where under appropriate reaction conditions known in the art, the addition of RNase H1 or RNase H2 enzyme will lead to cleavage of ‘n’ competitor polynucleotides at a position within the RNA bases or at the DNA-RNA junction when they are annealed to their fully complementary non-target sequence ‘n₁*’ and not when they remain unbound in single-stranded form or if they are annealed to a mismatched target sequence ‘n₁’ or other mismatched non-target sequence, and where the RNA domain is placed such that the 5′ cleavage product C1 remains bound following cleavage and the 3′ cleavage product C2 dissociates. and in the same reaction a DNA polymerase, with the inclusion of other reagents required for PCR amplification, the target sequences ‘n₁’ will be selectively amplified over the non-target sequences ‘n₁*’, where competitor cleavage on each ‘n₁*’ strand will lead to 3′ competitor cleavage product dissociation and enable a DNA polymerase to extend the 5′ competitor cleavage product and increase its melting temperature, and as the temperature is elevated, the uncleaved competitor on the ‘n₁’ strand will dissociate to enable the forward primer to extend on the ‘n₁’ target strand and ‘n₁’ PCR amplification can occur, where on each non-target ‘n₁*’strand, the stabilized extended competitor polynucleotide will remain annealed and prevent a DNA polymerase from extending the forward primer on the non-target ‘n₁*’ template and ‘n₁*’ PCR amplification will be suppressed.

In various aspects of the embodiment, the RNase H1 or RNase H2 cleavage and simultaneous PCR amplification are performed at multiple reaction temperatures following denaturation, the first of which enables annealing of the intact, uncleaved competitors at temperature t₁ and is followed by annealing and partial extension of the forward primers, annealing and extension of the reverse primers and at the same time competitor cleavage and extension on the T₁* non-target strands at temperature t₂, then when the temperature t₃ exceeds the annealing temperature of the intact competitors which subsequently dissociate from the T₁ target templates, allowing the forward primer to complete extension on the T₁ target strand but not the T₁* non-target strand due to the stabilized extended competitor.

In various embodiments, the allele enriched extendable competitor PCR involves a simultaneous detection step, or alternatively, a subsequent allele-specific qPCR is performed to detect the presence of target sequence T₁, or, the allelic enriched extendable competitor PCR is performed on an NGS library or an NGS library is simultaneously or subsequently made from the product of the allele enriched extendable competitor PCR and detection of target sequence T₁ is performed by NGS analysis.

In various aspects, any of the polynucleotide combinations provided herein comprise a modified nucleic acid. In various aspects, the competitor further comprises a modified nucleic acid, and in various embodiments of these aspects, the modified nucleic acid is in the PCR amplification primer pair and/or the modified nucleic acid is in the hairpin detection probe.

In each polynucleotide combination of the disclosure, various aspects are provided wherein the competitor comprises a plurality of modified nucleic acids, and wherein the PCR amplification primer pairs and hairpin detection probe comprise a plurality of modified nucleic acids.

In each polynucleotide combination, aspects are provided wherein the polynucleotide competitor further comprises a blocking group at its 3′ end which blocks extension from a DNA polymerase. In this aspect, an embodiment is provided wherein the blocking group is selected from the group consisting of a 3′ phosphate group, a 3′ amino group, a dideoxy nucleotide, a C3 spacer and an inverted deoxythymidine (dT).

In each polynucleotide combination, aspects are provided wherein the polynucleotide competitor further comprises a modified internucleotide linkage which blocks cleavage by RNase H1 or RNase H2. In this aspect an embodiment is provided wherein the nuclease resistant linkages are selected from the group consisting of a 2′-propoxyamine, 2′-methoxy, 2′-propoxy, 2′-methoxy-ethoxy, 2′-fluoro, phosphorothioate, methylene methylimino substitution of the phosphodiester linkage.

In each polynucleotide combination of the disclosure, aspects are provided wherein the amplification primer pair further comprises nuclease resistant linkages between bases at their 3′ ends or nuclease resistant nucleotides which block exonuclease cleavage by a proofreading DNA polymerase. In this aspect, an embodiment is provided wherein the nuclease resistant linkages are selected from the group consisting of a 3′ phosphorothioate and a 2′-O methyl RNA.

In each polynucleotide combination of the disclosure, aspects are provided wherein the hairpin detection probe further comprises modified nucleic acids that increase the binding affinity of the probe to its complementary sequence, the C1 competitor cleavage product. In this aspect, an embodiment is provided wherein the modified bases are selected from the group consisting of a locked nucleic acid (LNA), a minor groove binder (MGB), or a peptide nucleic acid (PNA).

In each aspect that the polynucleotide combination provides, embodiments include those wherein the hairpin detection probe comprises a label. In various aspects, the label is located in the hairpin detection probe at its 3′ end and/or the label is quenchable. In various aspects of these embodiments, the hairpin detection probe also comprises a quencher and/or the quencher is located at the junction of the single-stranded domain and the double-stranded hairpin domain. In specific embodiments, the quencher is selected from the group consisting of Black Hole Quencher 1, Black Hole Quencher-2, Iowa Black FQ, Iowa Black RQ, and Dabcyl. G-base.

The preceding summary of the subject matter of the disclosure is supplemented by the following description of various aspects and embodiments of the disclosure, as provided in the following enumerated paragraphs.

Paragraph 1. A composition comprising a first polynucleotide and a second polynucleotide, wherein: (A) the first polynucleotide comprises a sequence such that: (i) the first polynucleotide has a fully complementary domain to a target polynucleotide (T1) such that the first polynucleotide is able to hybridize to T1 under appropriate conditions, and the sequence comprises a RNA base that is susceptible to cleavage by a ribonuclease when the RNA base is hybridized to T1; and (ii) the first polynucleotide is mismatched to a non-target polynucleotide region (T1*) at the position of the RNA base or 1, 2 or 3 nucleotides adjacent to the RNA base; and (iii) T1* is a sequence variant of T1; and (B) the second polynucleotide comprises a sequence such that: (iv) the second polynucleotide is fully complementary to a target polynucleotide region (T2) and a non-target polynucleotide region (T2) that overlaps T1 and T1* by at least one nucleotide, wherein T2 is upstream of T1 and T1*.

Paragraph 2. The composition of paragraph 1, wherein the RNA base on the first polynucleotide is located at the midpoint of the first polynucleotide.

Paragraph 3. The composition of paragraph 1 or paragraph 2, further comprising at least one additional RNA base on the first polynucleotide located immediately adjacent to the first RNA base.

Paragraph 4. The composition of paragraph 3 wherein the first polynucleotide comprises at least 4 consecutive RNA bases.

Paragraph 5. The composition of paragraph 4 wherein one or more RNA bases are susceptible to cleavage by a ribonuclease when the polynucleotide is hybridized to the target sequence T1.

Paragraph 6. The composition of paragraph 5 wherein modified nucleotides at one or more RNA bases renders the one or more bases resistant to cleavage by a ribonuclease.

Paragraph 7. The composition of paragraphs 1-6 wherein the 3′ terminus of the first polynucleotide is blocked from initiation of extension by a DNA polymerase.

Paragraph 8. The composition of any one of paragraphs 1-7 wherein the first polynucleotide comprises a detectable marker and a moiety that quenches the detectable marker.

Paragraph 9. The composition of paragraph 8 wherein the detectable marker and the moiety are on opposite sides of the RNA base, and in a configuration that prevents detection of the detectable marker.

Paragraph 10. The composition of paragraph 9 wherein cleavage of the first polynucleotide results in detection of the detectable marker.

Paragraph 11. The composition of any one of paragraphs 1-10 wherein T2 overlaps T1 and T1* by at least about 1 to at least about 50 nucleotides.

Paragraph 12. The composition of any one of paragraphs 1-11 wherein the ribonuclease includes but is not limited to RNase H2 or RNase H1.

Paragraph 13. A method of initiating polymerase extension on a target polynucleotide in a sample using the composition of any one of paragraphs 1-12; wherein the sample comprises a target polynucleotide that comprises (i) a sequence T1 in a first region that is fully complementary to the sequence of a domain in the first polynucleotide; and (ii) a sequence T2 that is fully complementary to the sequence in the second polynucleotide; the method comprising the step of (a) contacting the sample with the composition and a polymerase under conditions that allow extension of a sequence from T2 following cleavage and dissociation of the first polynucleotide.

Paragraph 14. A method of amplifying a target polynucleotide in a sample using the composition of any one of paragraphs 1-12, wherein: the sample comprises a mixture of (i) a target polynucleotide having a sequence in a first region (T1) that is fully complementary to the sequence of a domain in the first polynucleotide, and a sequence in a second region (T2) that is fully complementary to the sequence in the second polynucleotide; and (ii) one or more non-target polynucleotides that are not fully complementary to T1; the method comprising the steps of: (a) contacting the sample with the composition and a polymerase under conditions that allow extension of a sequence (S) from T2, wherein the sequence is complementary to the target polynucleotide when the target polynucleotide is present in the sample; (b) denaturing the sequence (S) extended from T2 from the target polynucleotide, and (c) repeating step (a) in the presence of a third polynucleotide having a sequence complementary to a region (T3) in the sequence extended from T2 in step (b) to amplify the target polynucleotide, wherein extension and amplification of the target polynucleotide to generate a product occurs when the first polynucleotide is fully complementary to the sequence in T1, but is less efficient or does not occur when the first polynucleotide is not fully complementary to the sequence in a non-target sequence T1*; and wherein steps (a)-(c) are followed by further extension and amplification of the product when the first polynucleotide hybridizes to T1 or the second polynucleotide hybridizes to T2, and the third polynucleotide hybridizes to T3 in the presence of the polymerase.

Paragraph 15. The method of paragraph 14, further comprising: (iii) a fourth polynucleotide having a sequence that is fully complementary to a region T4 in a second target polynucleotide in the sample, such that the fourth polynucleotide is able to hybridize to T4 under appropriate conditions, and the sequence comprises a RNA base that is susceptible to cleavage by a ribonuclease when the RNA base is hybridized to T4; and (iv) a fifth polynucleotide having a sequence that is fully complementary to a region T5 in a second target polynucleotide in the sample, wherein T5 overlaps T4 and T4* by at least one nucleotide, and wherein T5 is upstream of T4 and T4*; wherein (v) the fourth polynucleotide is mismatched to a non-target polynucleotide region (T4*) at the position of the RNA base or 1, 2 or 3 bases adjacent to the RNA base; and wherein the sample comprises a mixture of (i) a target polynucleotide having a sequence in a first region (T4) that is fully complementary to the sequence of a domain in the fourth polynucleotide, and a sequence in a second region (T5) that is fully complementary to the sequence in the fifth polynucleotide; and (ii) one or more non-target polynucleotides that are not fully complementary to T4.

Paragraph 16. The method of any one of paragraphs 13-15 further comprising the step of adding a ribonuclease at step (a).

### DETAILED DESCRIPTION OF THE DRAWINGS

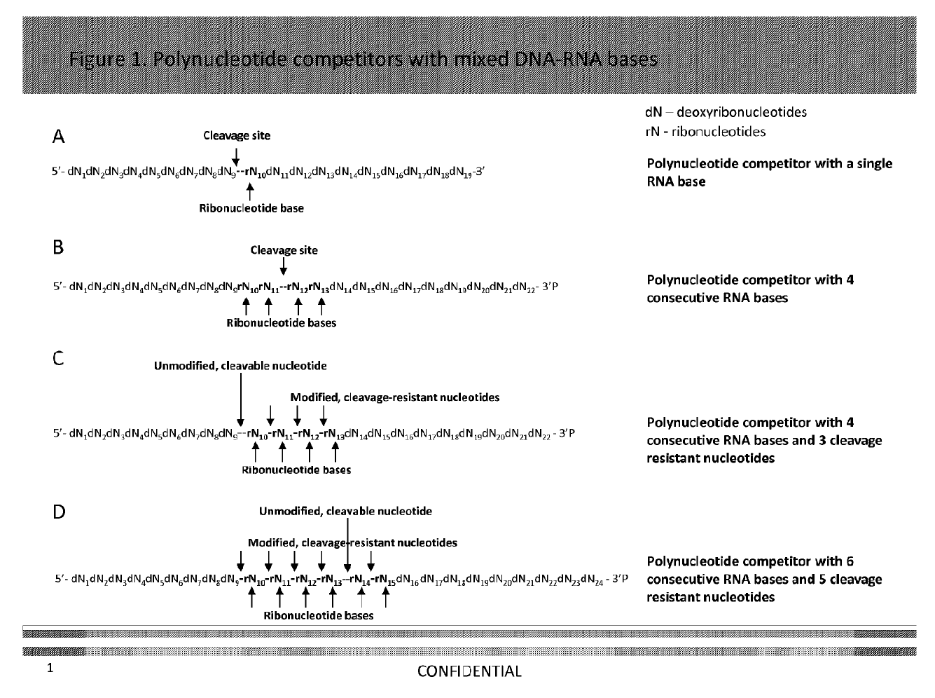

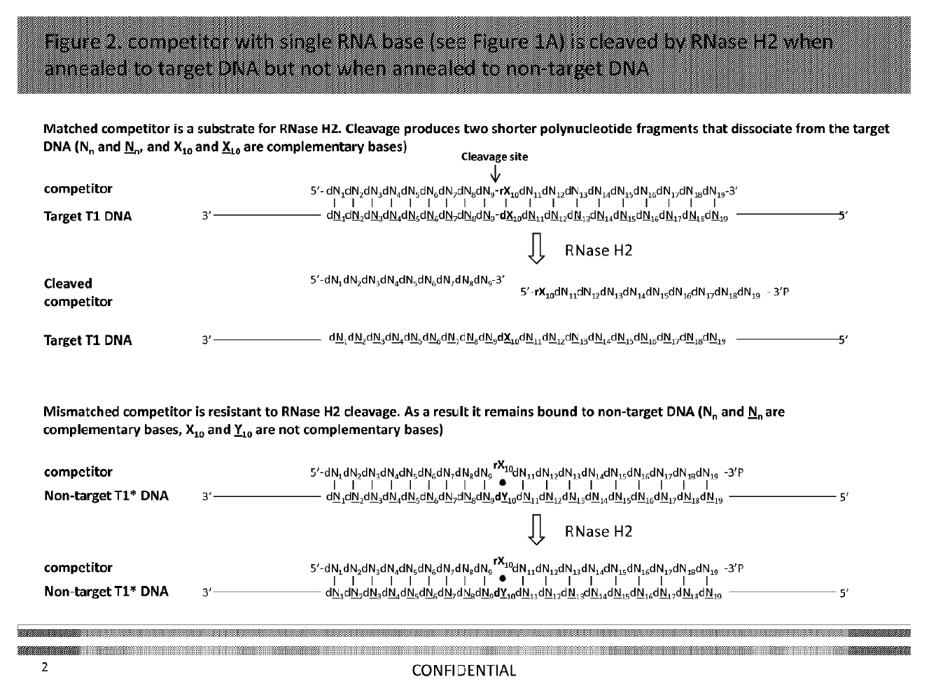

FIG. 1 depicts various example compositions of a cleavable competitor polynucleotide.

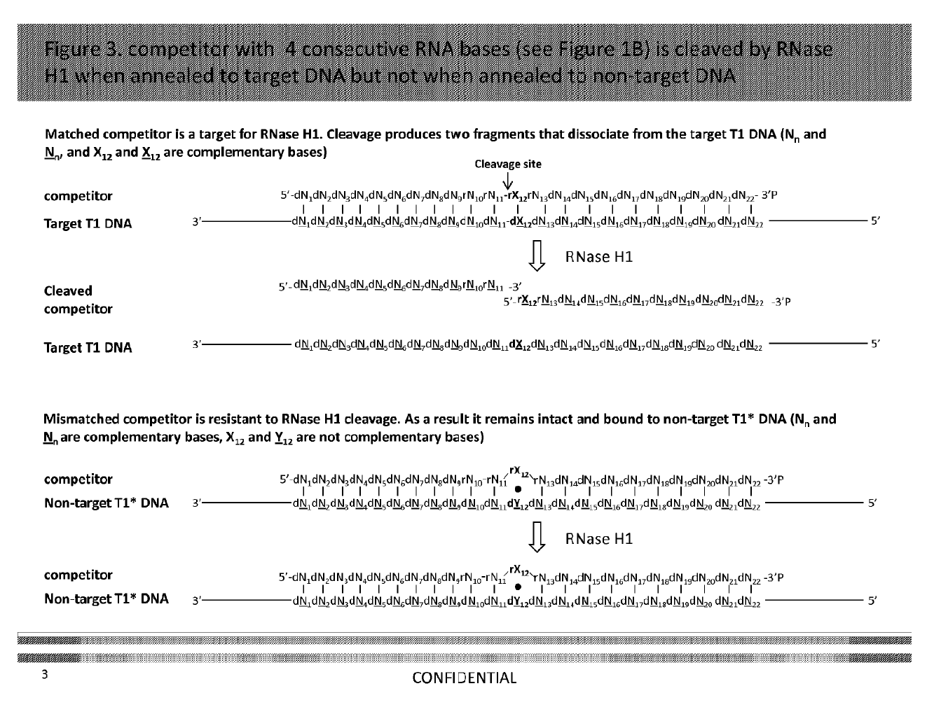

FIG. 2 depicts annealing of a cleavable competitor to its matched target (T1) and to its mismatched non-target (T1*) templates, where RNase H2 cleavage occurs only on the fully complementary target template.

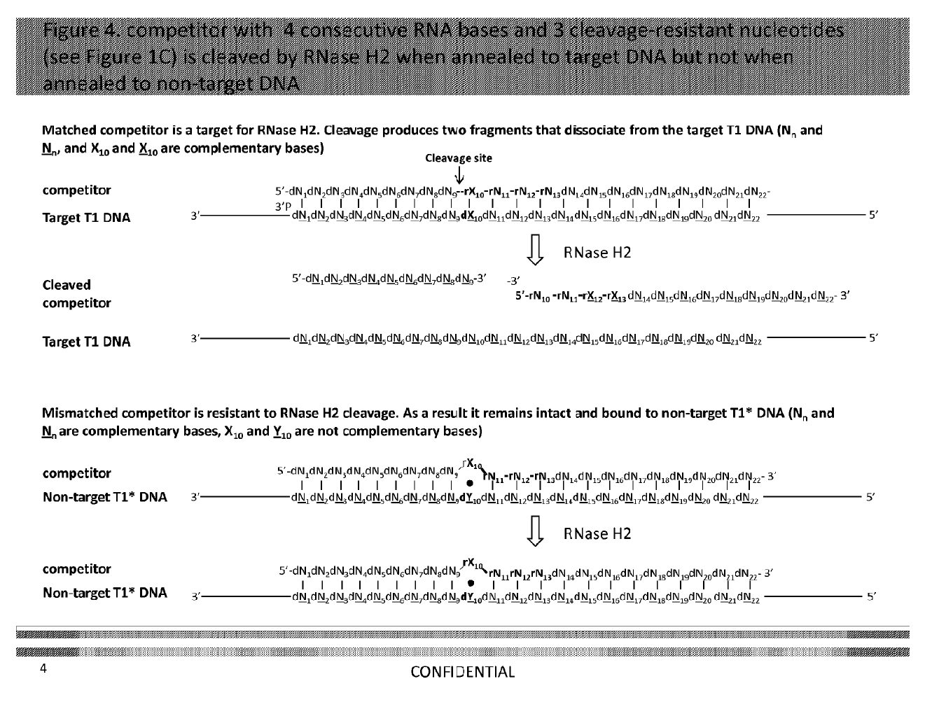

FIG. 3 depicts how RNase H1 enzyme cleaves the matched hybrid on the target sequence T1 whereas the mismatched non-target hybrid T1* is cleavage-resistant.

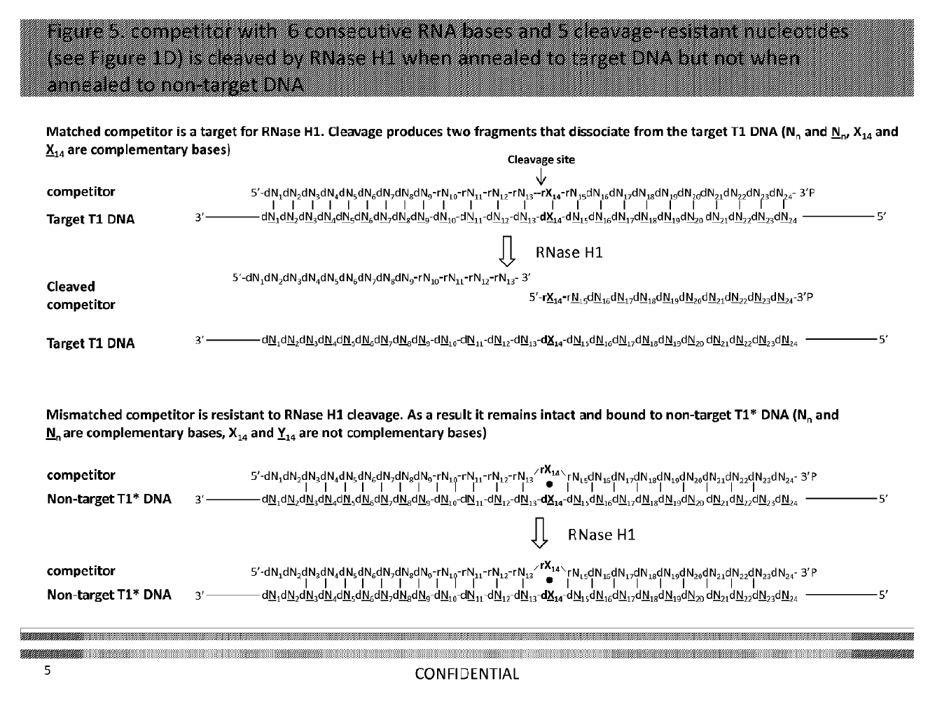

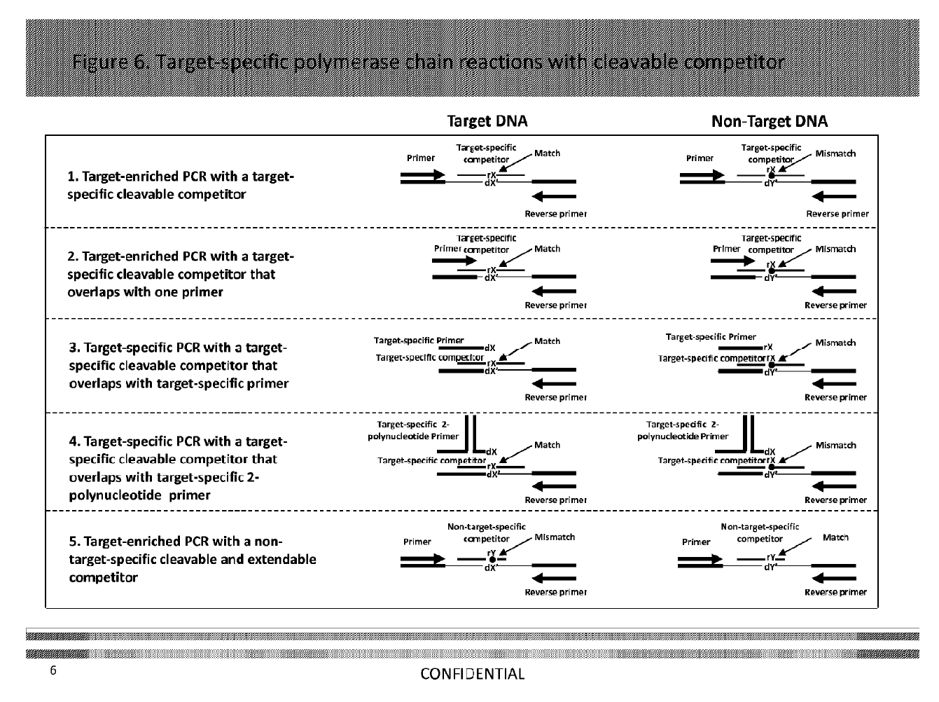

FIG. 4 depicts how RNase H2 enzyme cleaves the matched hybrid on the target sequence T1 whereas the mismatched non-target hybrid T1* is cleavage-resistant.

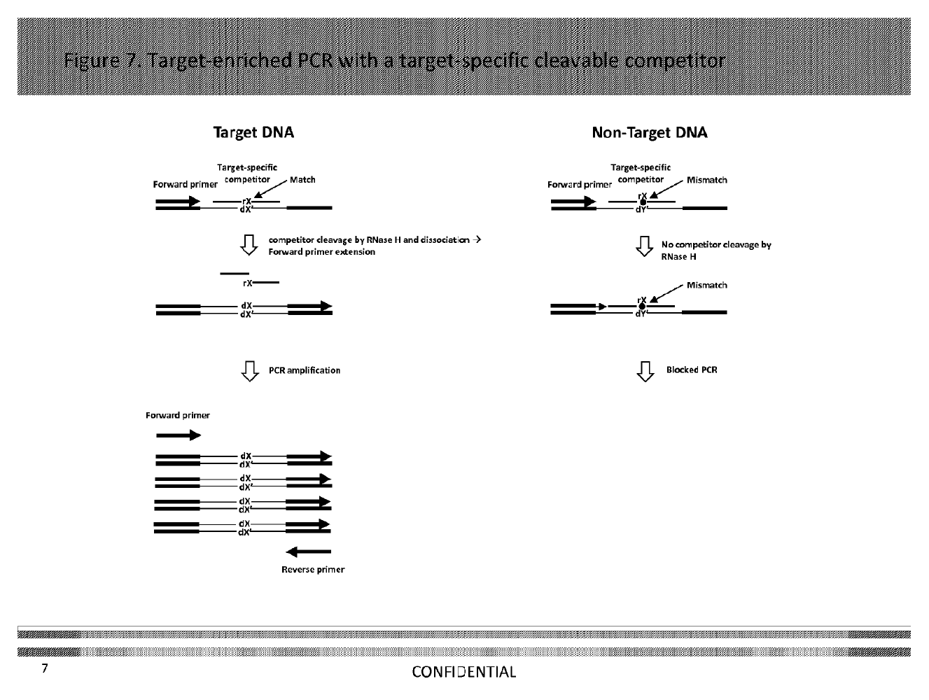

FIG. 5 depicts how RNase H1 enzyme cleaves the matched hybrid on the target sequence T1 whereas the mismatched non-target hybrid T1* is cleavage-resistant.

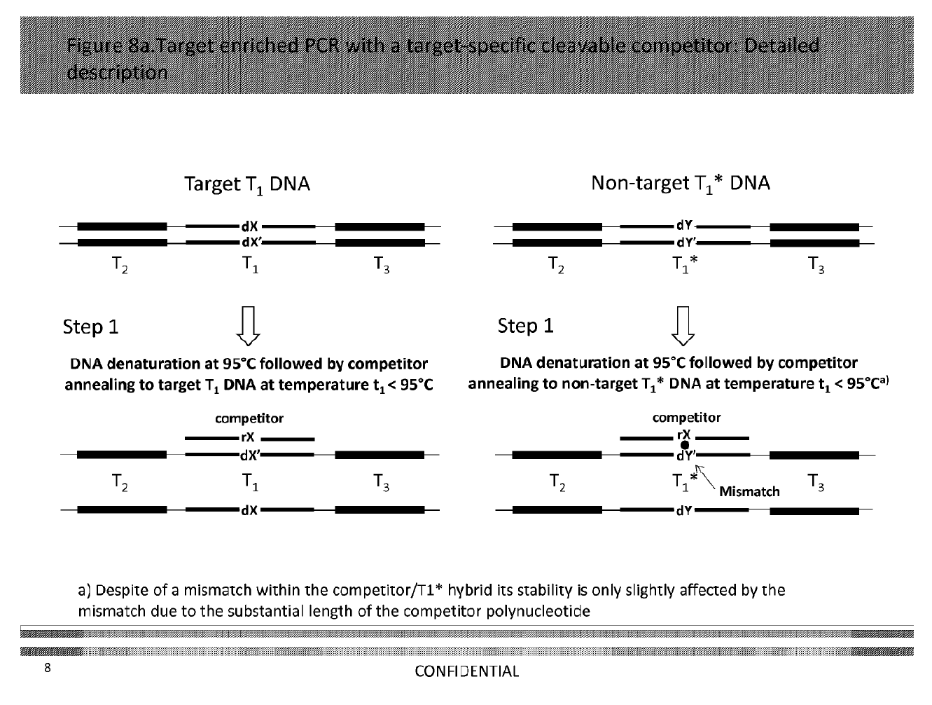

FIG. 6 depicts various cleavable competitor and primer combinations disclosed herein.

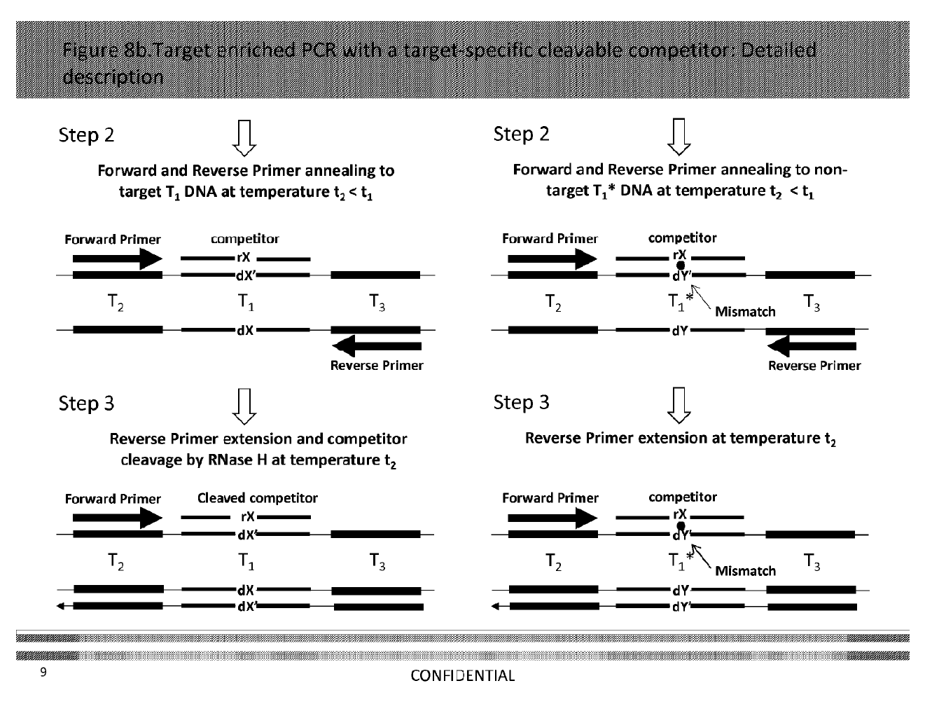

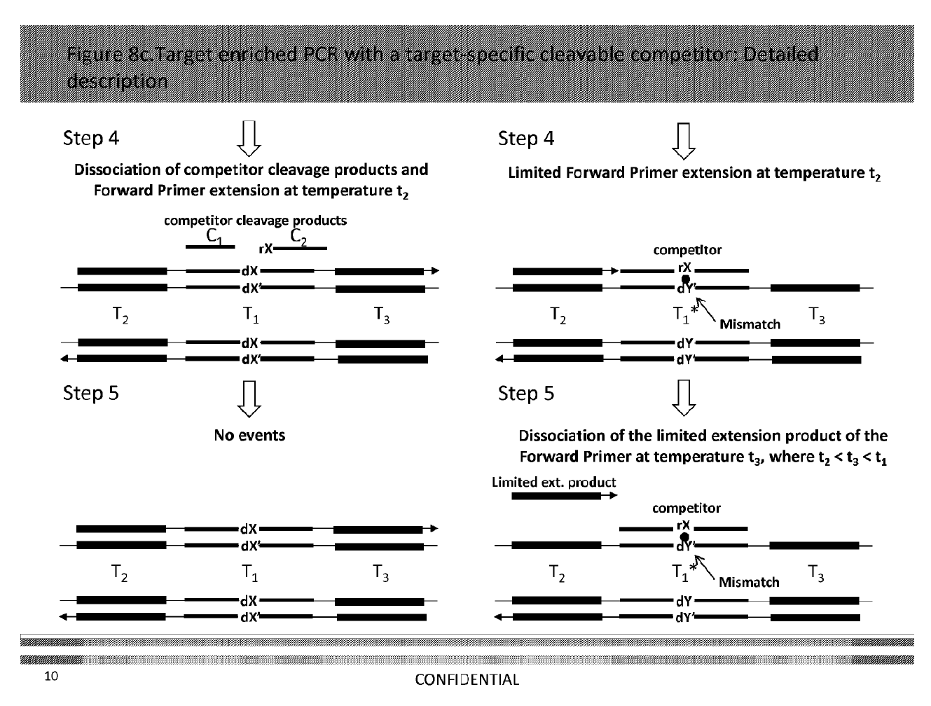

FIG. 7 depicts how target-enriched PCR amplification can be achieved using a cleavable polynucleotide competitor.

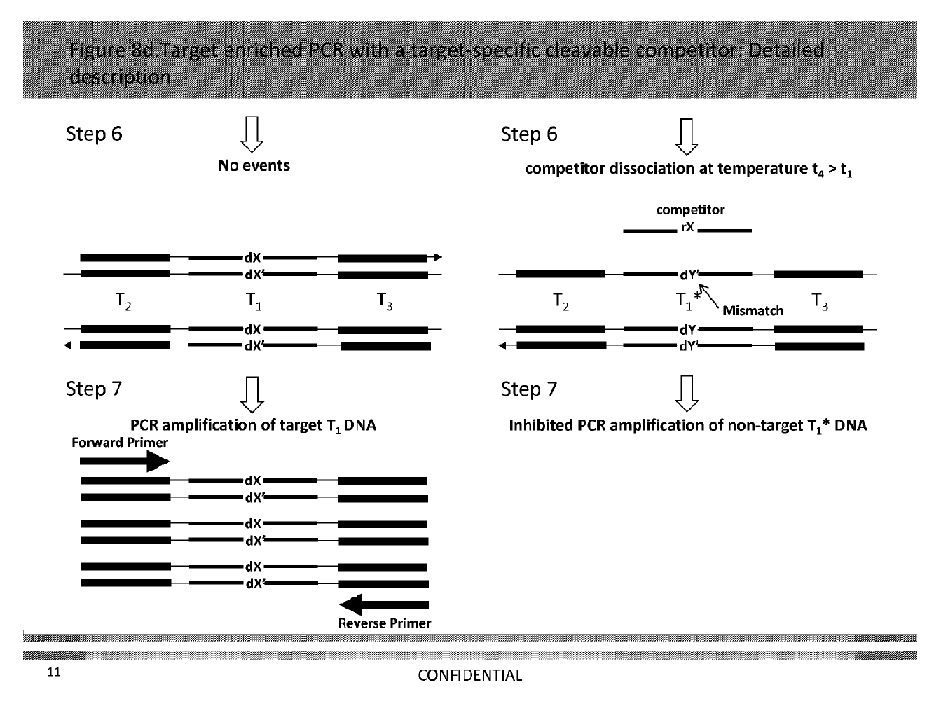

FIG. 8a-d provide a detailed description of how target-enriched PCR amplification can be achieved using a cleavable polynucleotide competitor.

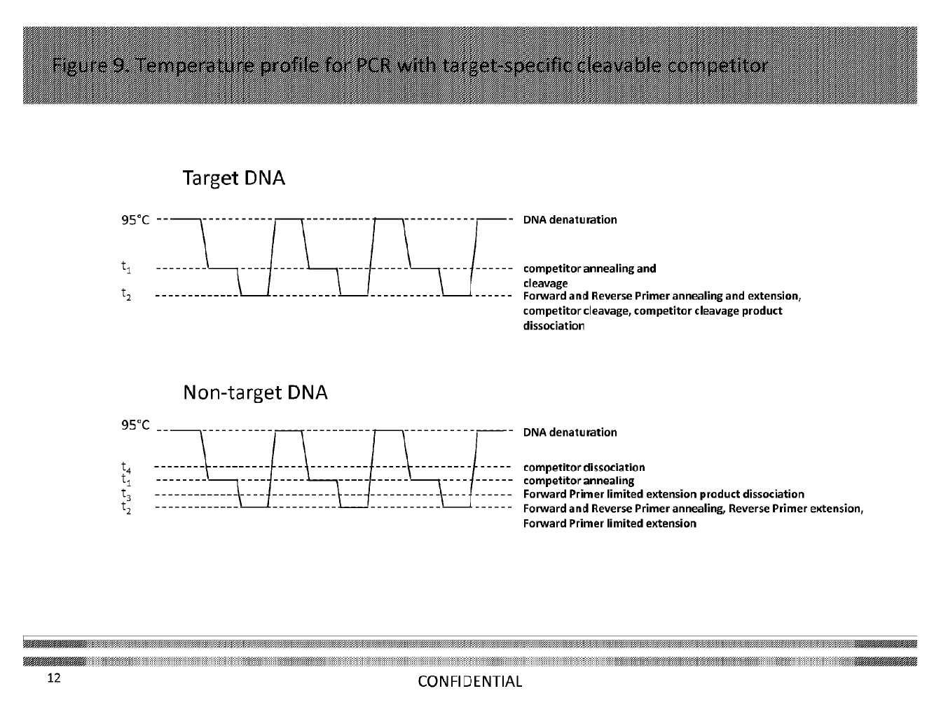

FIG. 9 depicts the thermocycling profile for the example outlined in FIGS. 7 and 8.

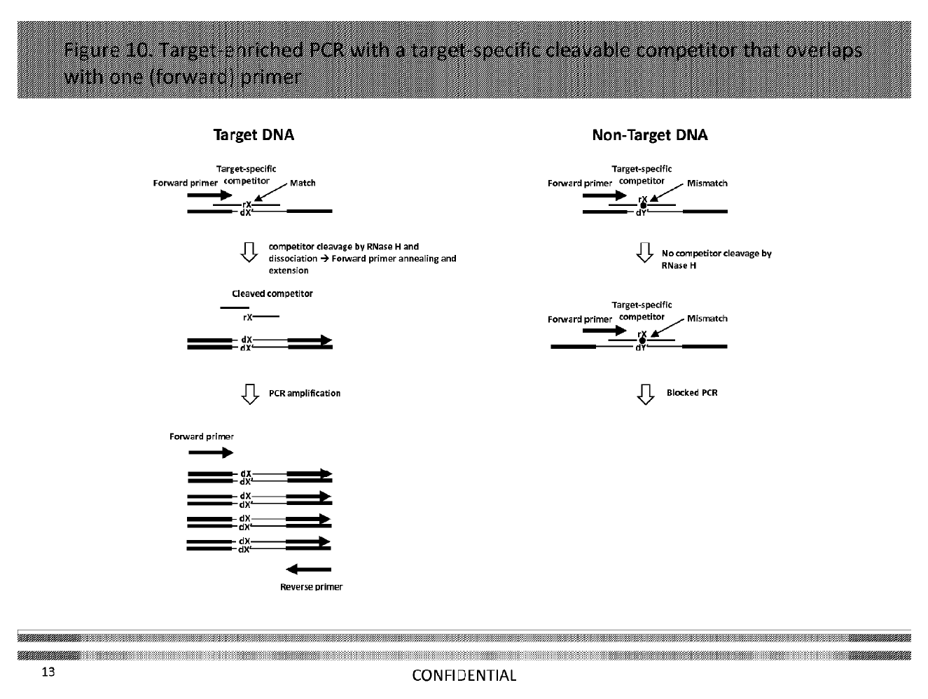

FIG. 10 depicts how target-enriched PCR amplification can be achieved using a cleavable polynucleotide competitor and overlapping forward primer.

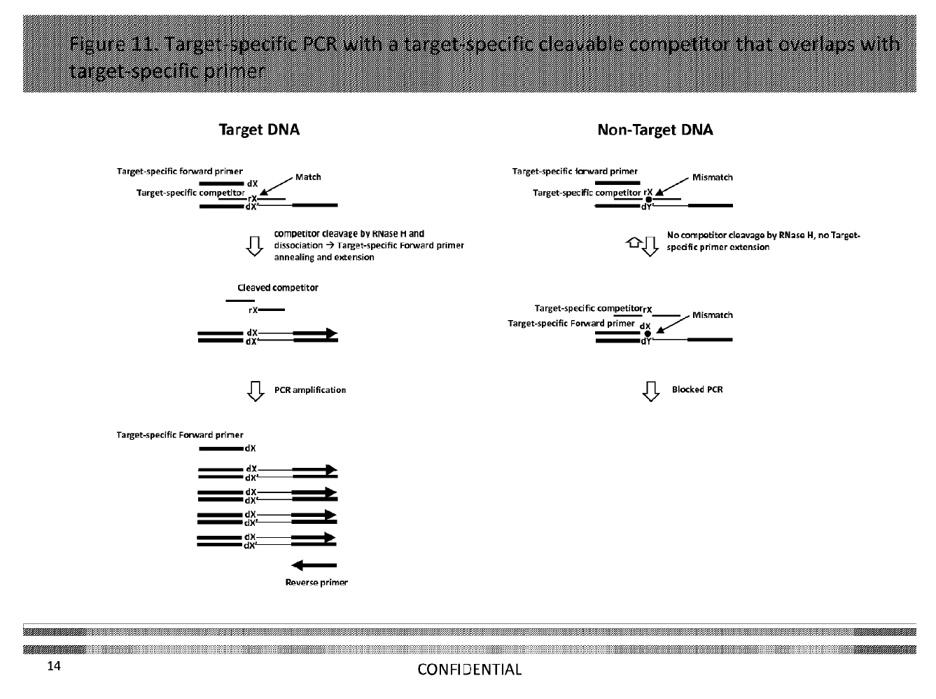

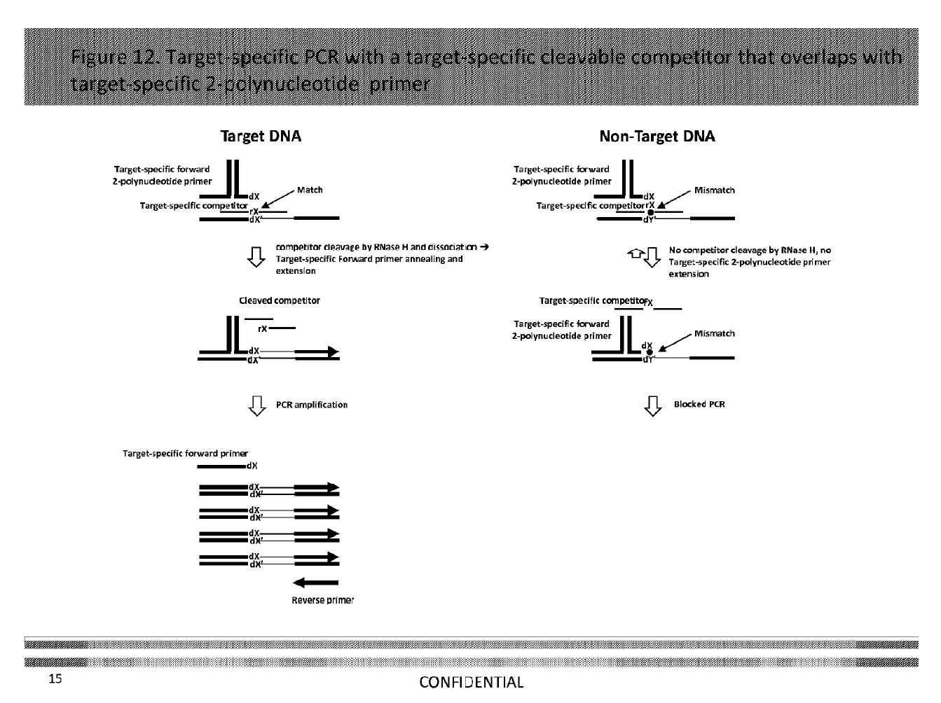

FIG. 11 depicts how target-specific PCR amplification can be achieved using a cleavable polynucleotide competitor and an overlapping target-specific forward primer.

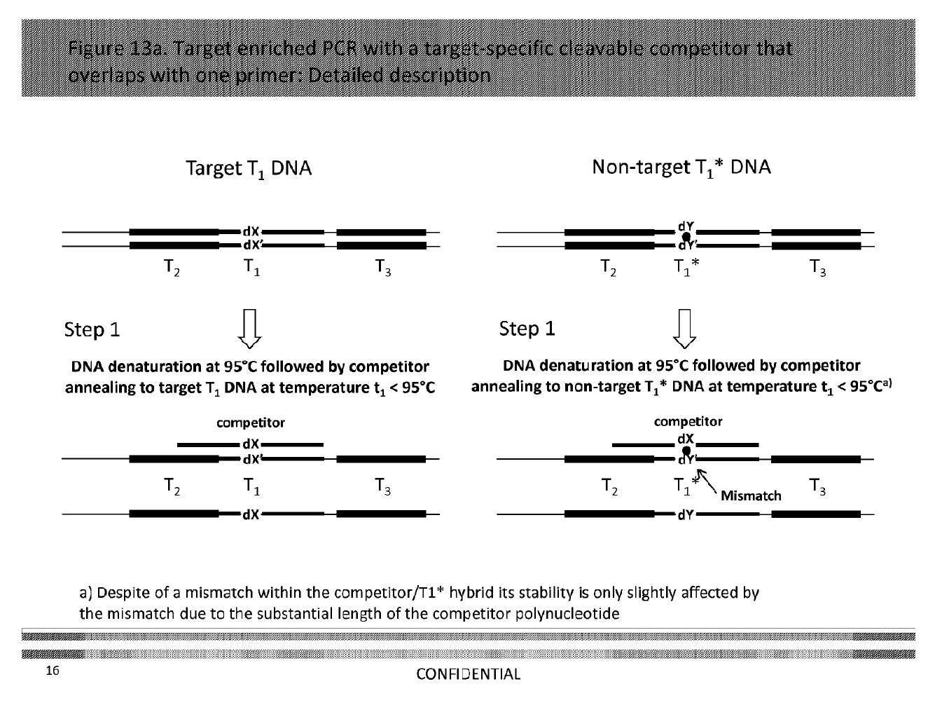

FIG. 12 depicts how target-specific PCR amplification can be achieved using a cleavable polynucleotide competitor and an overlapping target-specific 2-polynucleotide primer.

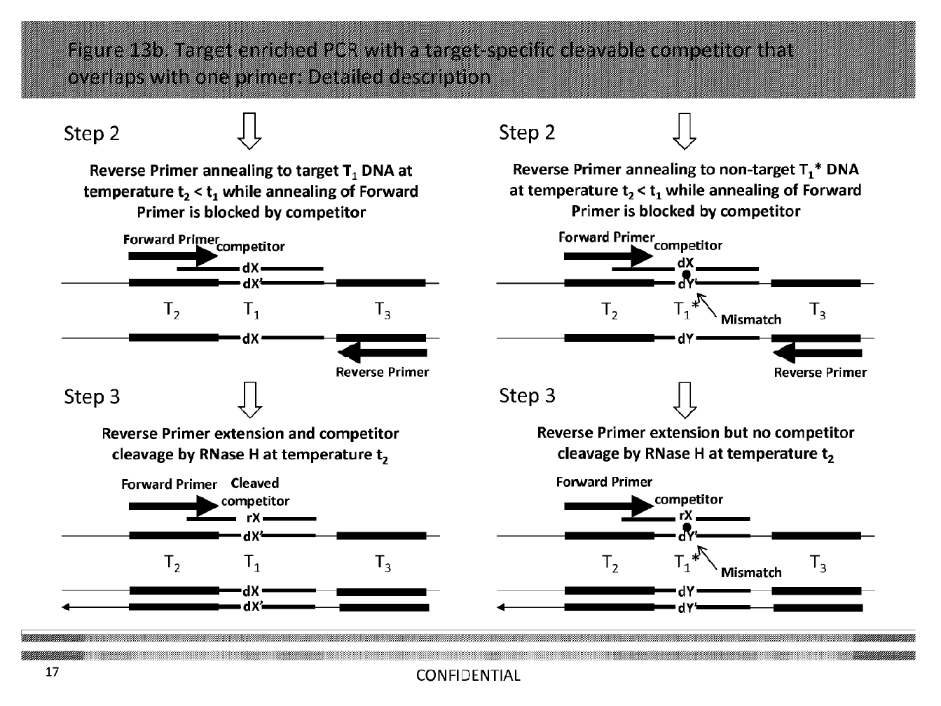

FIG. 13a-d provide a detailed description of how target-enriched PCR amplification can be achieved using a cleavable polynucleotide competitor and an overlapping forward primer.

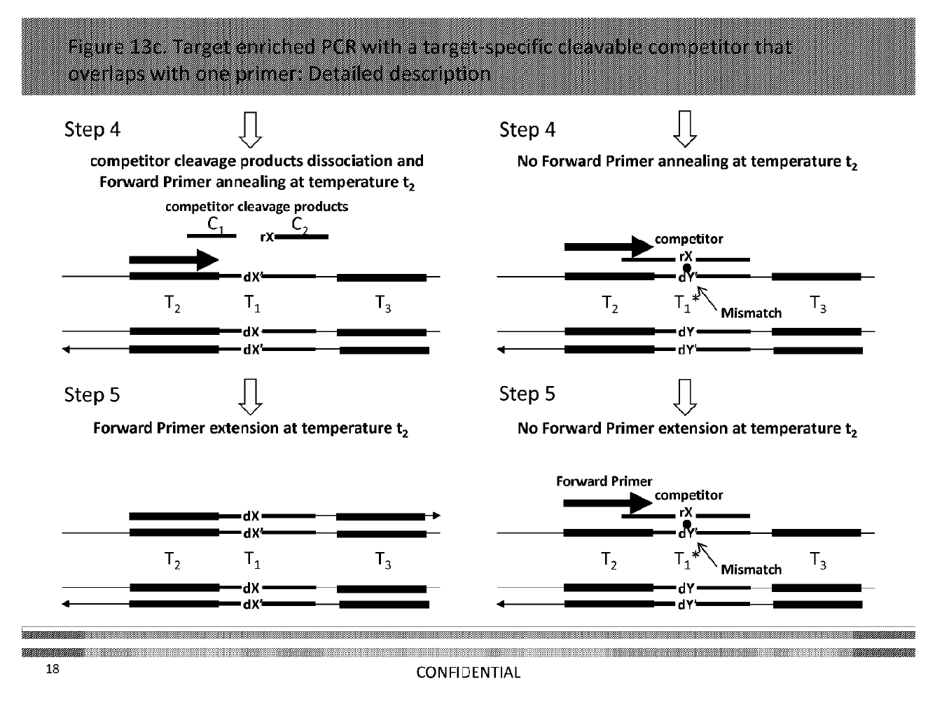

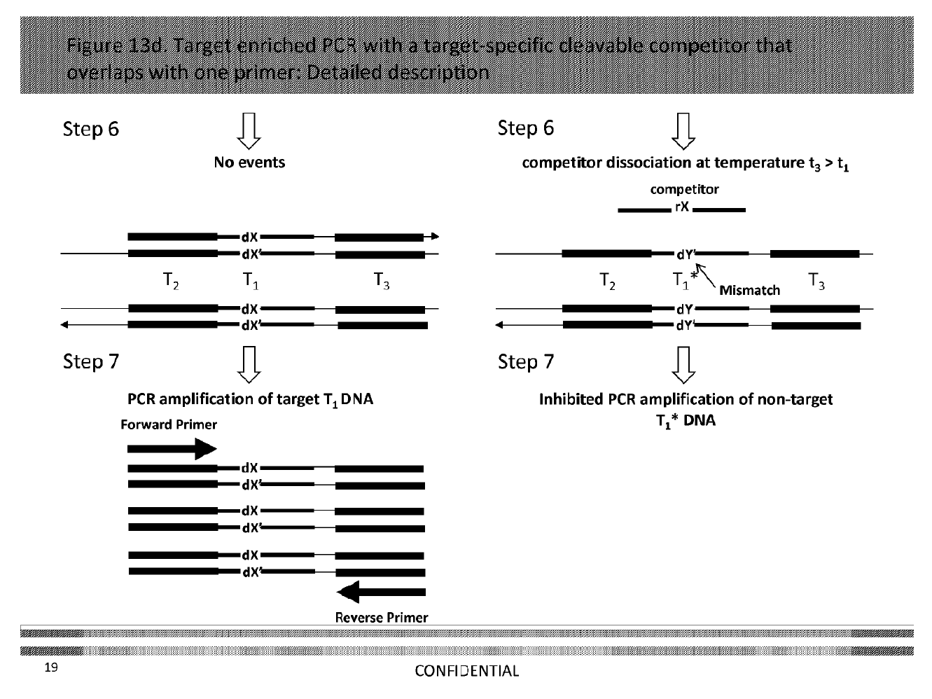

FIG. 14 depicts the thermocycling profile for the example outlined in FIGS. 10 and 13.

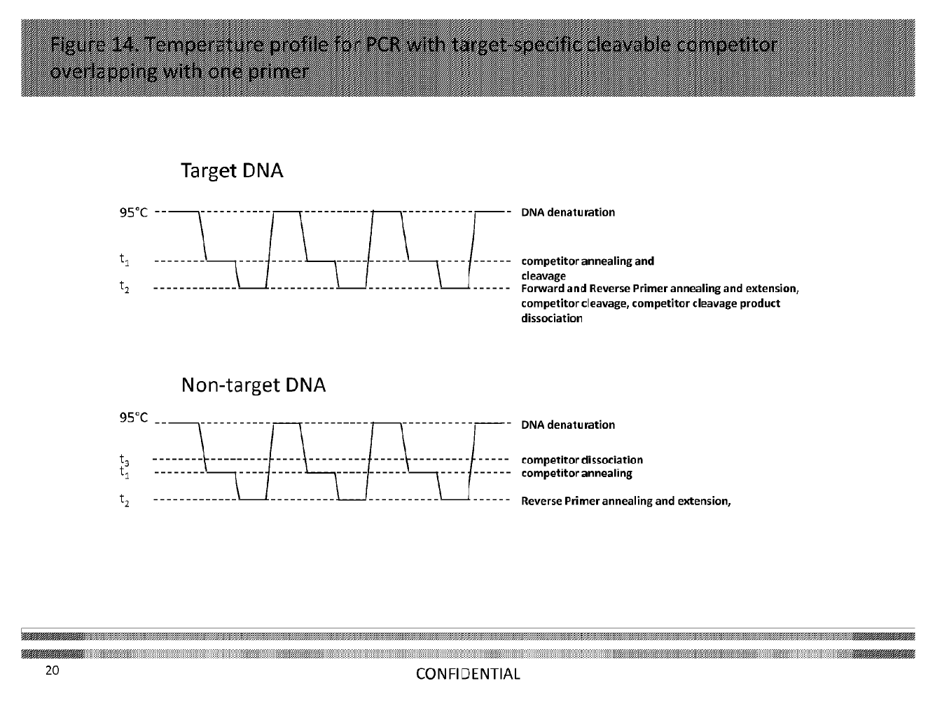

FIG. 15 depicts target-enriched PCR using a non-target specific extendable cleavable competitor.

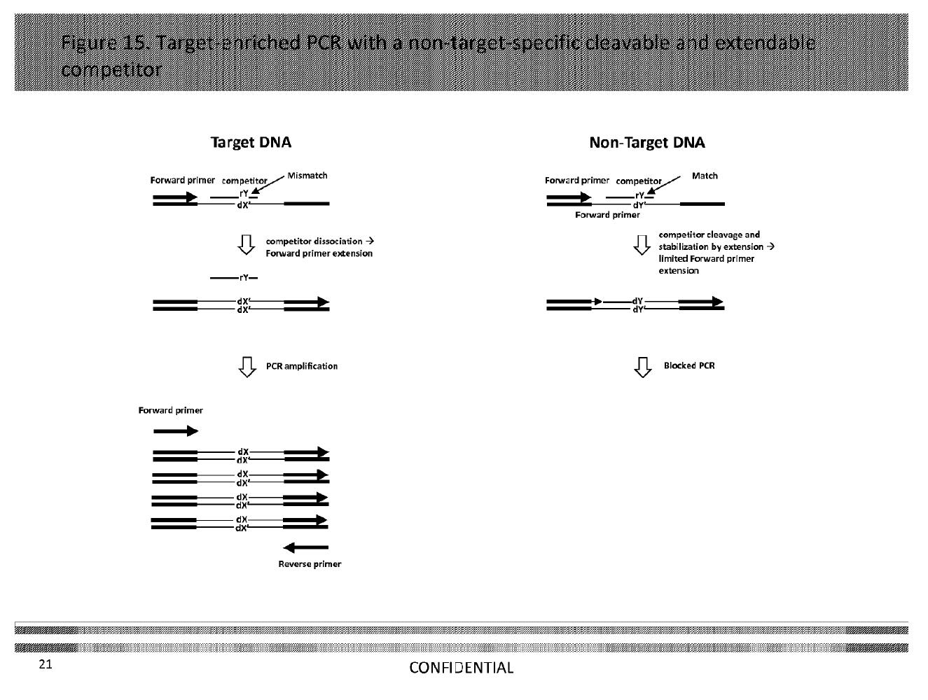

FIG. 16a-d provides a detailed description of how target-enriched PCR can be achieved using a non-target specific extendable cleavable competitor.

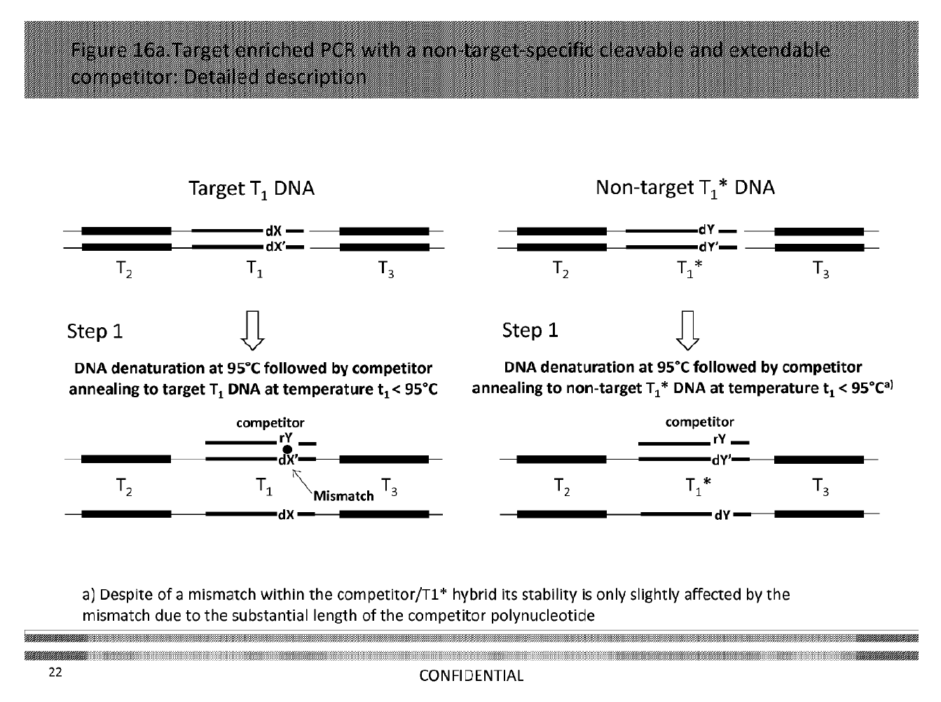

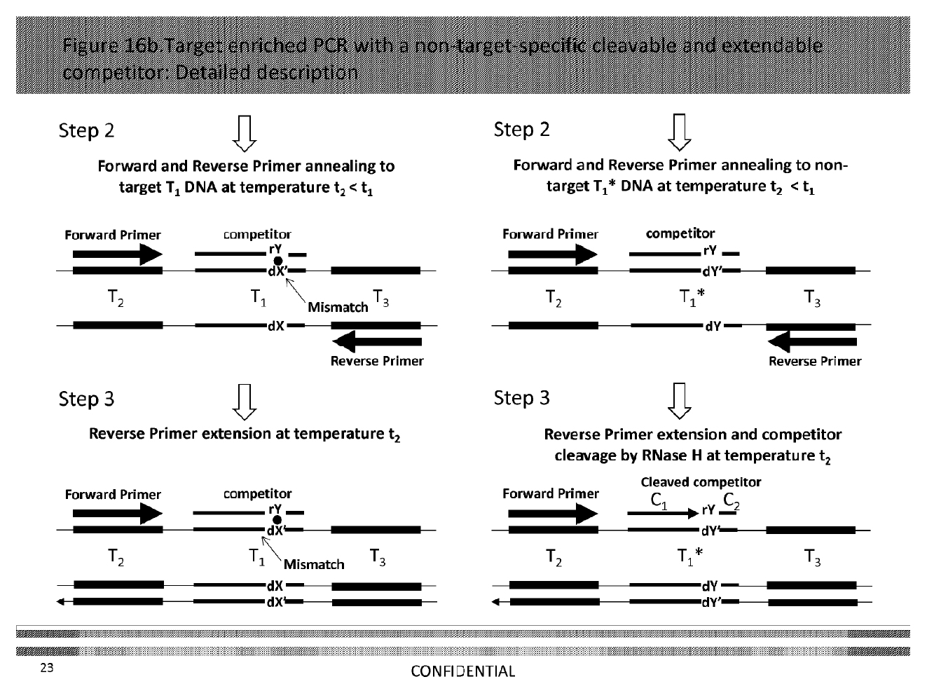

FIG. 17 depicts a thermocycling profile for the method outlined in FIGS. 15 and 16.

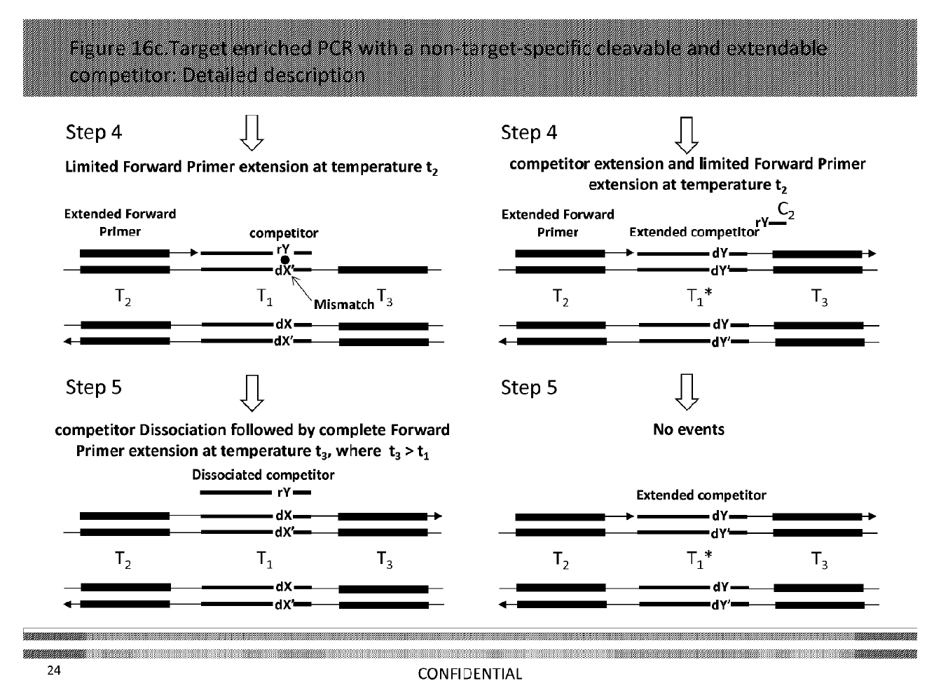

FIG. 18 depicts multiplexed target-enriched PCR amplification for 3 target sequences in an NGS library.

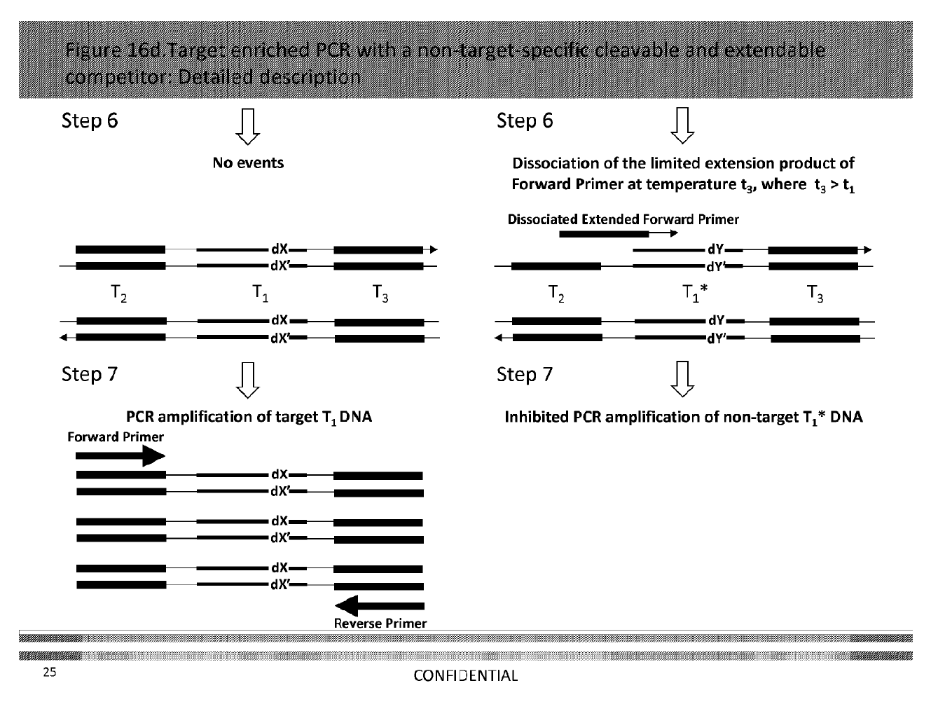

FIG. 19 depicts multiplexed target-enriched PCR amplification for ‘n’ target sequences in an NGS library.

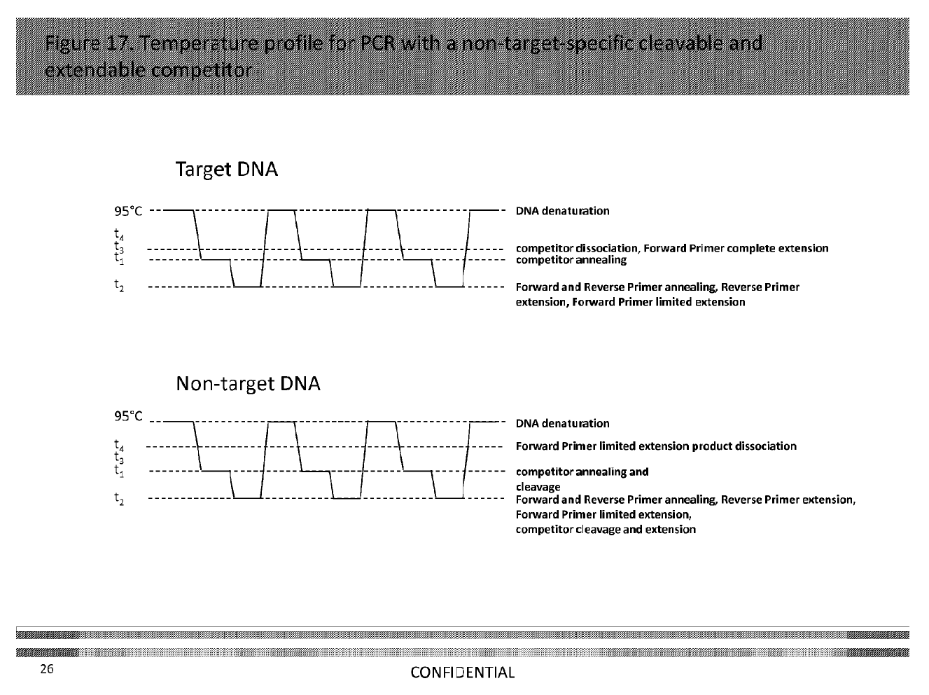

FIG. 20 depicts how competitor cleavage product C1 anneals and extends on the hairpin probe to generate a detectable signal.

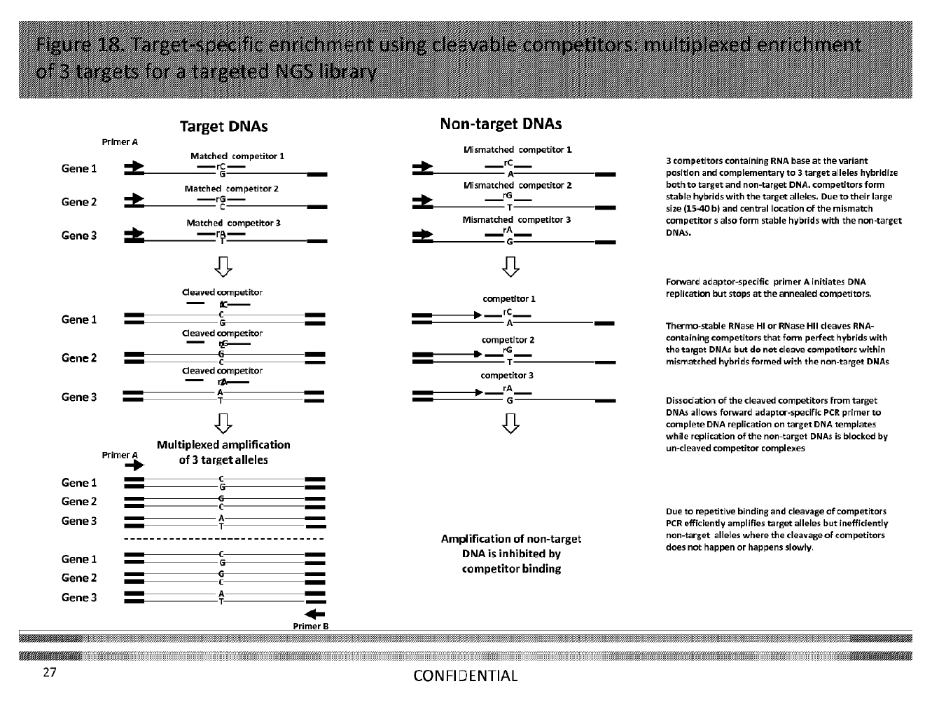

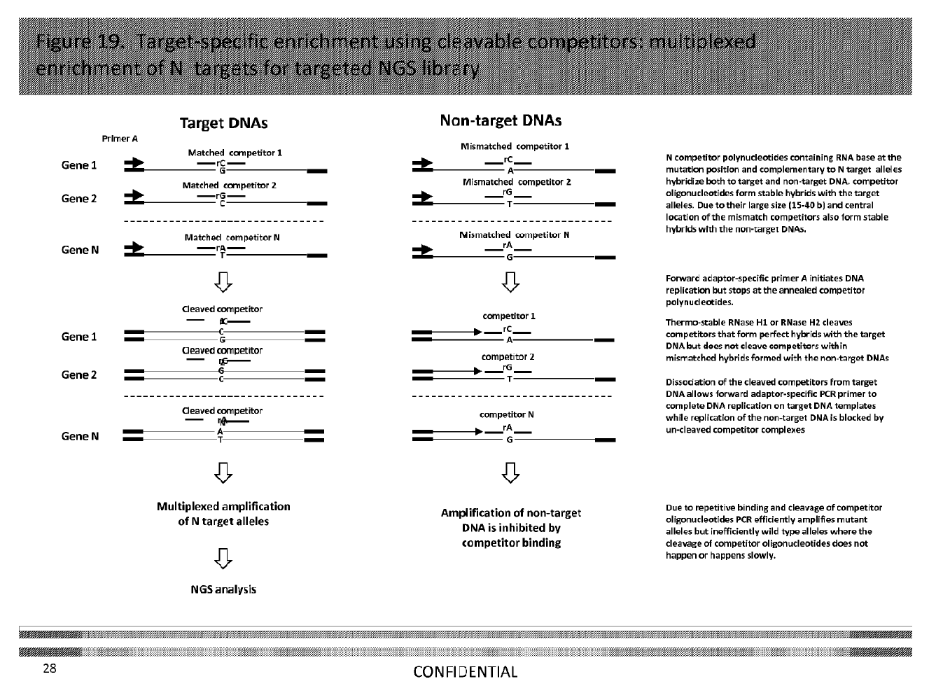

FIG. 21 depicts target-specific PCR amplification combined with hairpin probe detection.

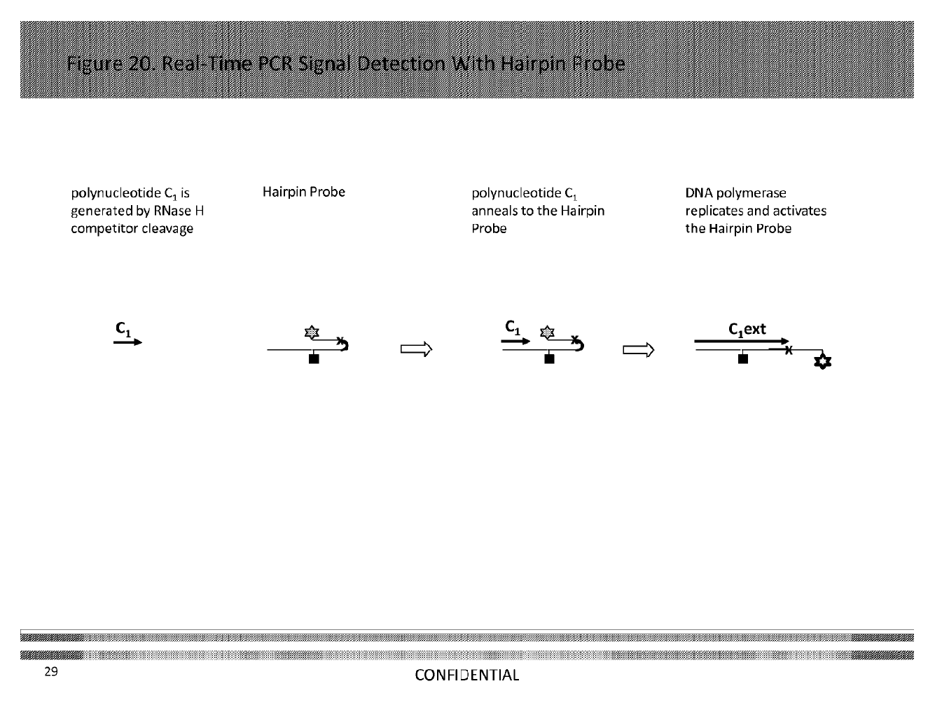

FIG. 22 depicts how a hairpin detection probe can be incorporated onto a portion of the competitor polynucleotide when it is designed to cleave on the target strand T₁.

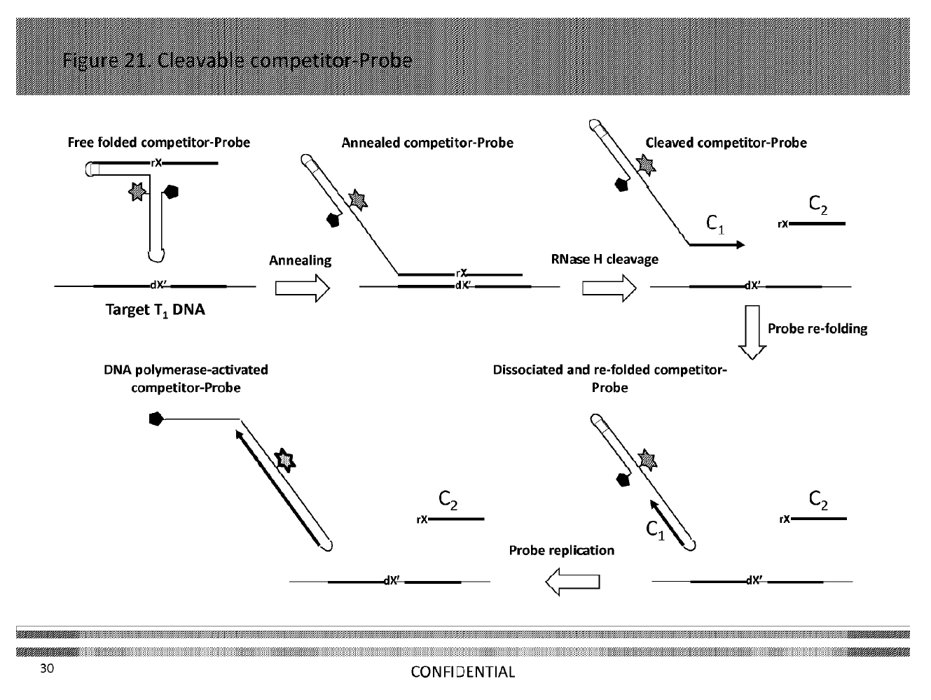

FIG. 23 depicts an NGS amplicon where adaptor sequence A overlaps with the Competitor sequence.

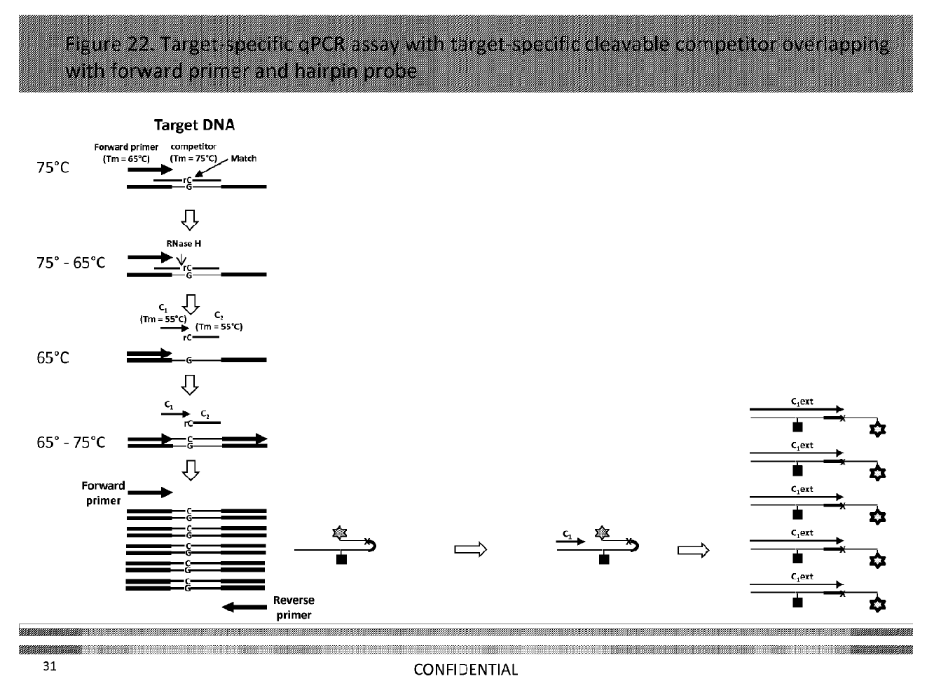

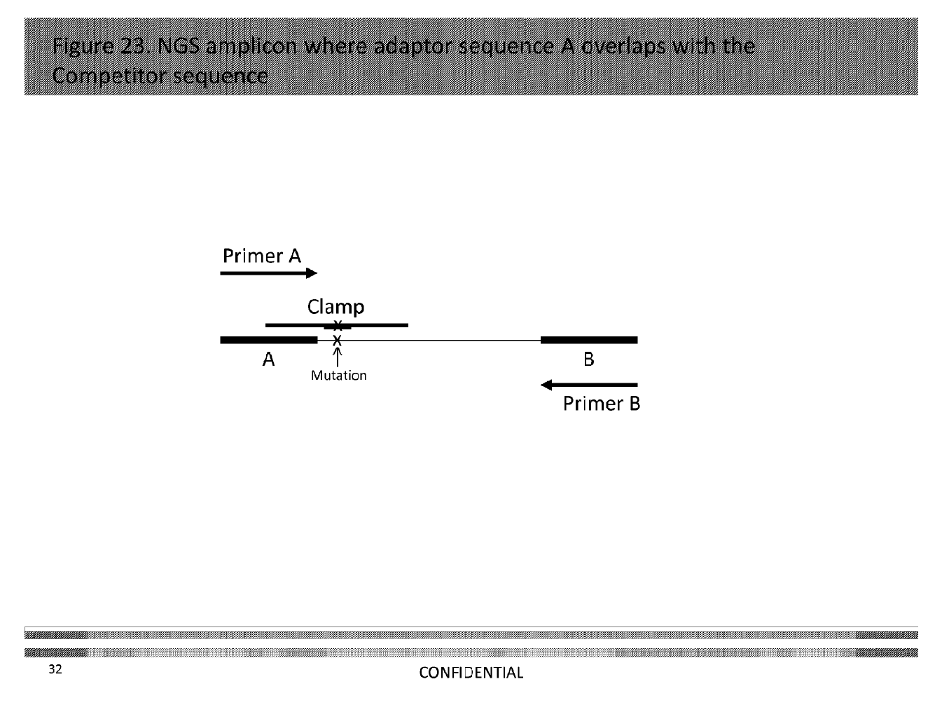

FIG. 24 depicts a Competitor with a 5′ non-genomic domain to increase stability of the C1/Hairpin Probe interaction.

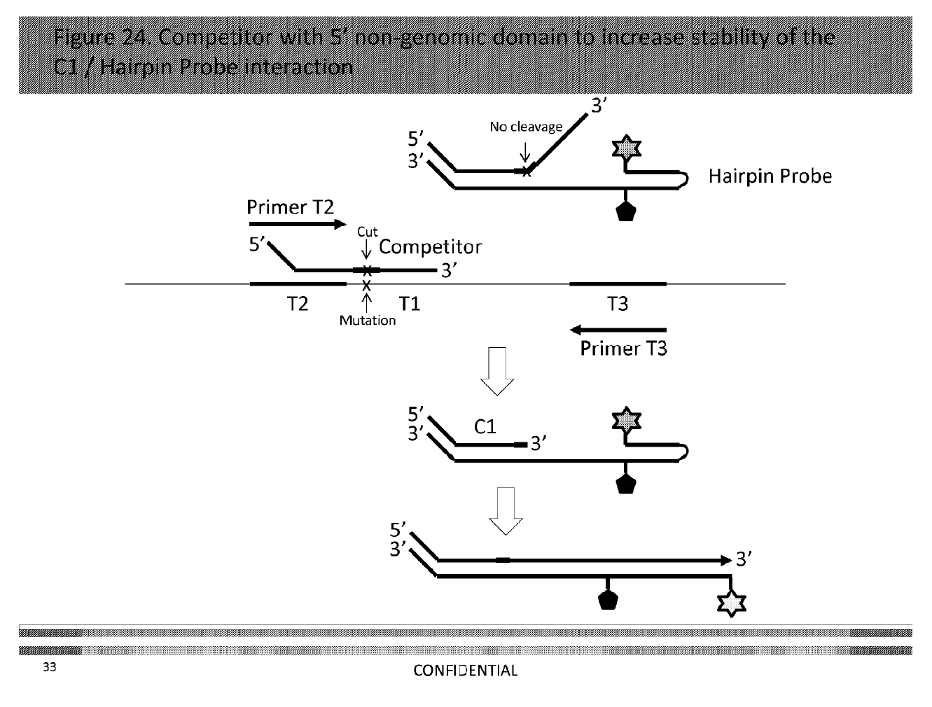

FIG. 25 depicts a Cleavable Competitor with a 5′ non-genomic domain and Cleavable Probe.

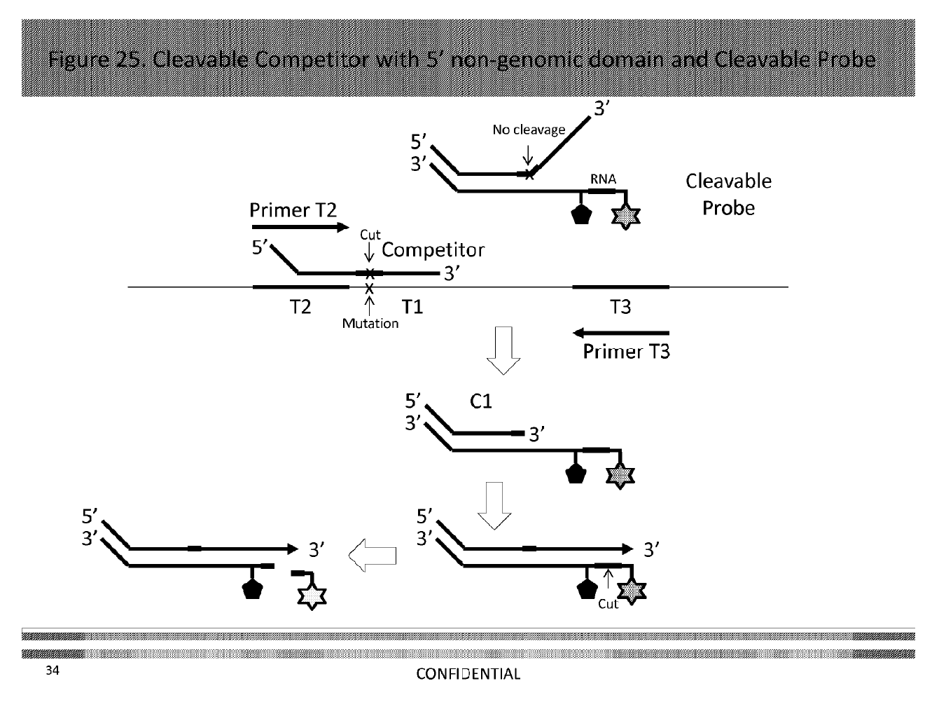

FIG. 26 depicts a Cleavable Competitor as a Cleavable Probe.

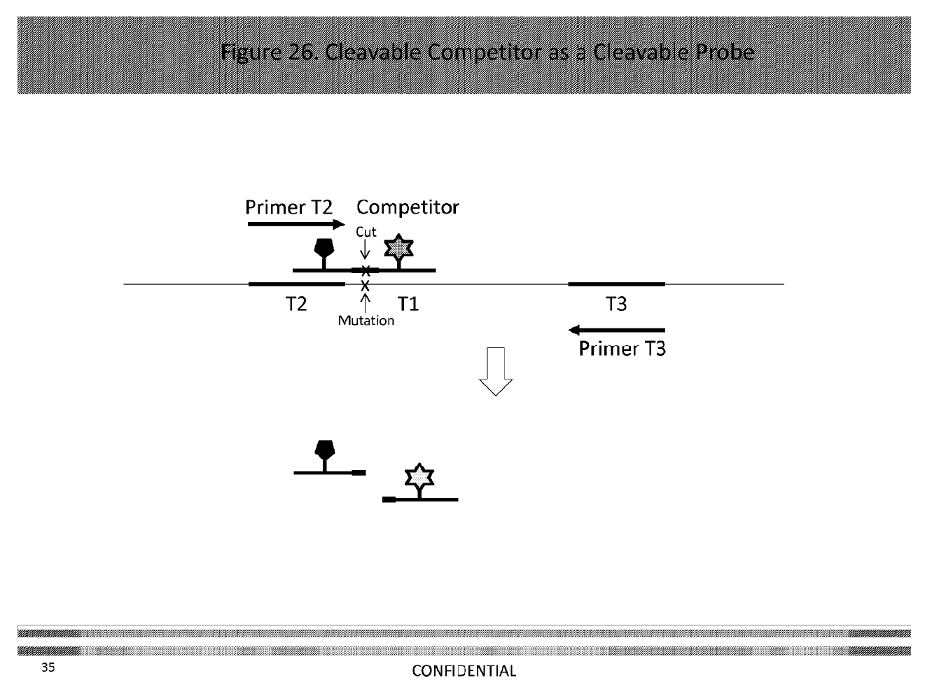

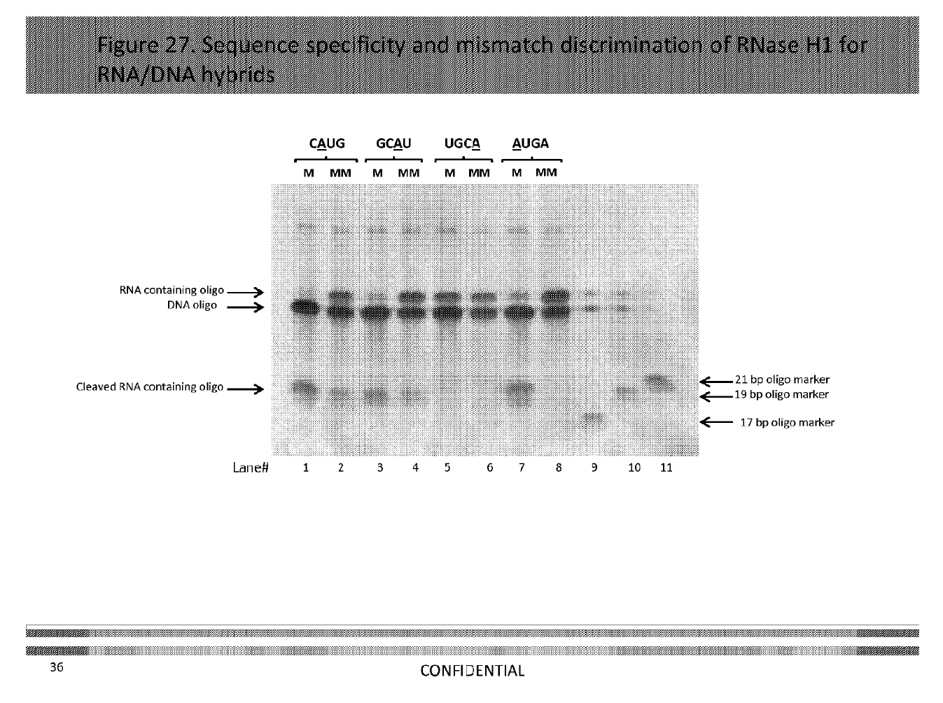

FIG. 27 shows the sequence specificity of RNase H1 for a RNA/DNA hybrid for a sequence with efficient cleavage versus a sequence with inefficient cleavage. It also demonstrates the mismatch discrimination ability of RNase H1 as it cleaves the matched hybrids much more efficiently than mismatched hybrids.

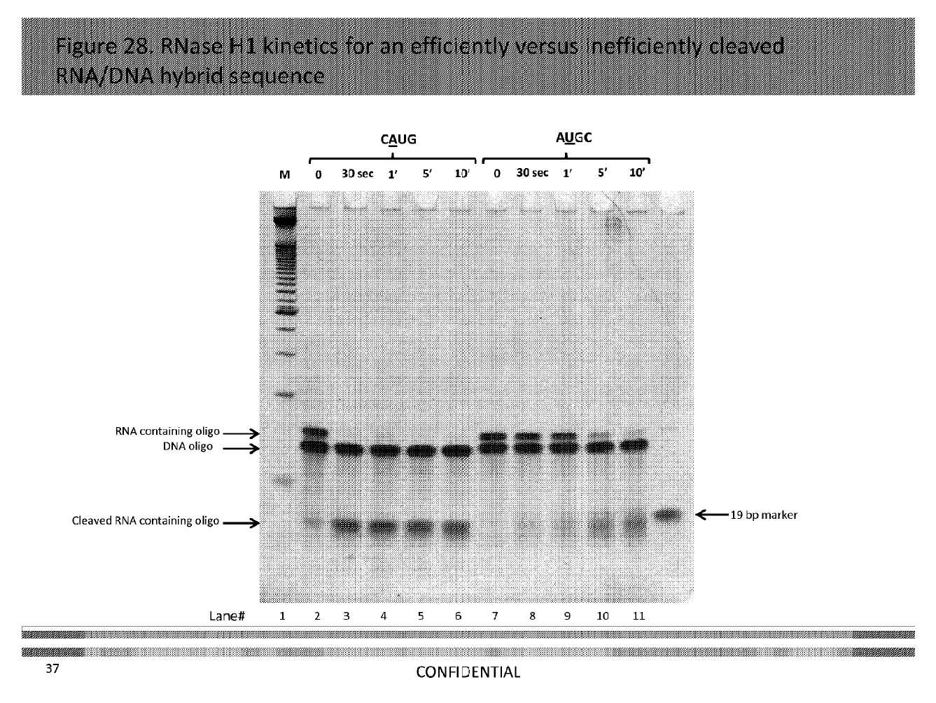

FIG. 28 depicts the kinetics of cleavage of RNase H1 for an RNA/DNA hybrid sequence with efficient cleavage versus an RNA/DNA hybrid sequence with inefficient cleavage.

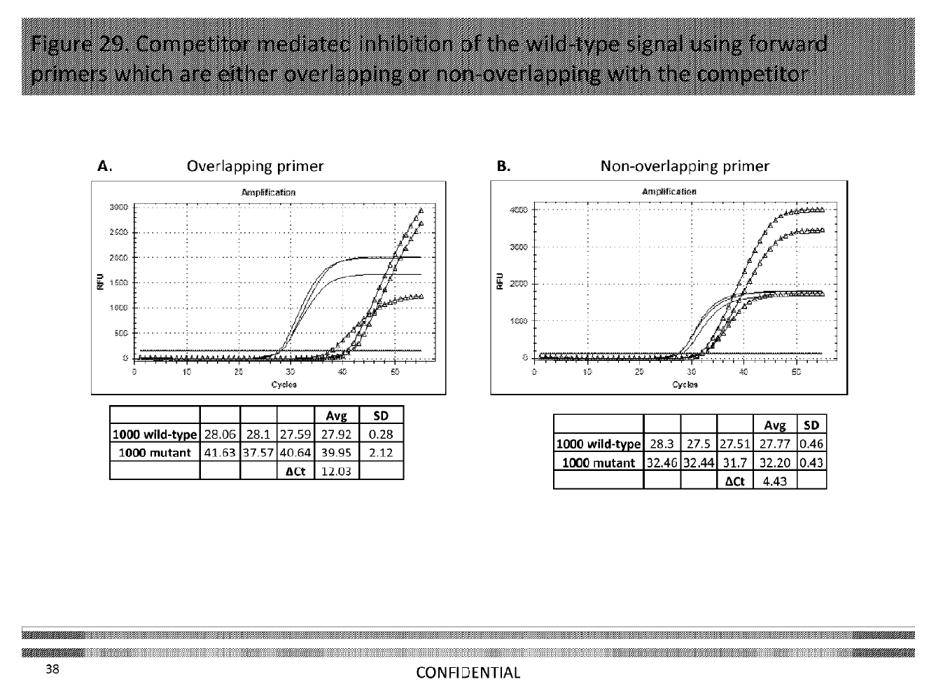

FIG. 29 depicts how an overlapping primer is better than a non-overlapping primer in terms of inhibition of the wild-type signal with a competitor.

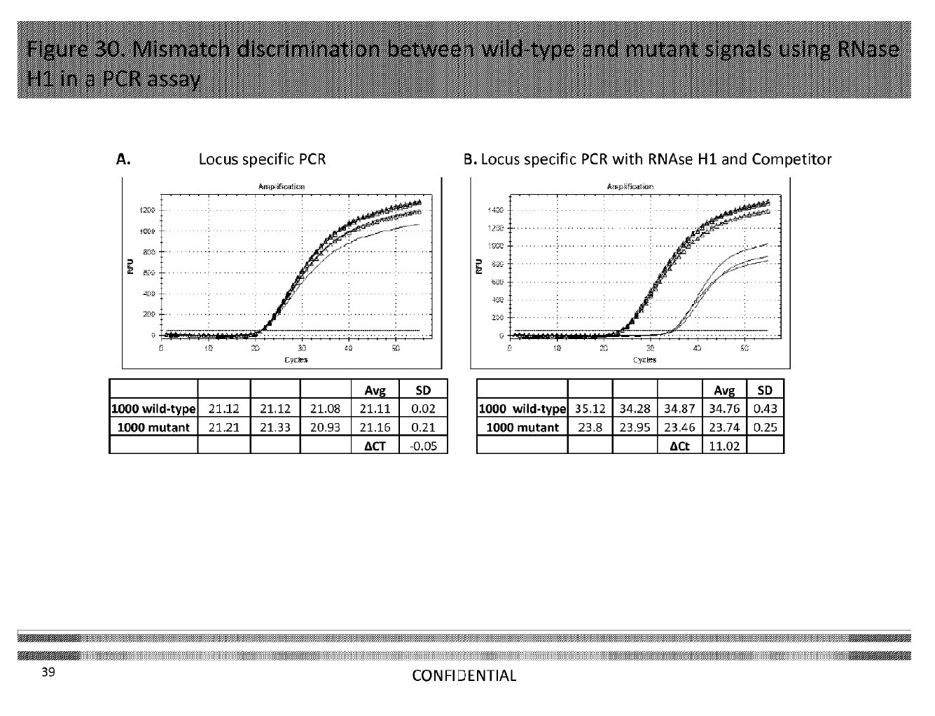

FIG. 30 depicts how competitor and RNase H1 can be used in PCR to discriminate between wild type and mutant templates.

### DETAILED DESCRIPTION OF THE INVENTION

The disclosure is based on the discovery of a method by which RNase H1 and RNaseH2 can be used to enrich or amplify target DNA molecules and prevent amplification of non-target DNA molecules. The disclosure also provides a method to multiplex various target DNA molecules in a single tube with high sensitivity and specificity. The disclosure further provides the use of RNAseH1 and H2 for a novel detection method using hairpin shaped probes which can be used in qPCR to quantify amplified target DNA. These aspects are useful in qPCR and NGS diagnostic assays.

The sensitivity of existing qPCR and NGS technologies are typically limited to 1% and 5% respectively, which is not sufficient for rare allele detection. Another limitation with the current qPCR assays is the inability to combine multiple mutation detection assays into one multiplex diagnostic assay. Multiple mutation detection requires multiple primers and probes which can cause either non-specific amplification of DNA or lead to formation of primer-dimers which greatly reduces the efficiency of a qPCR assay. There thus remains a need for the development of allelic enrichment and amplification strategies which will enable detection of rare mutations with high sensitivity and specificity. There also remains a need for development for single tube multiplex diagnostic assays which can greatly reduce the cost and time per assay. RNase H1 and H2 based cleavage assays described herein offer an advantage over current qPCR and NGS assays by overcoming these limitations thus addressing the need of the current market.

As used herein, “fully complementary” means that two polynucleotides share 100% complementarity over the full length nucleotide sequence of any of the polynucleotides disclosed herein. By way of nonlimiting example, a cleavable competitor that is 20 nucleotides in length is fully complementary to a target polynucleotide if all 20 nucleotides can base pair with a region of the target polynucleotide.

### I. Polynucleotides

As used herein, the term “polynucleotide,” either as a component of a polynucleotide combination, including cleavable competitor polynucleotides, primers and probes, or as a target molecule, is used interchangeably with the term oligonucleotide.

The term “nucleotide” or its plural as used herein is interchangeable with modified forms as discussed herein and otherwise known in the art. In certain instances, the art uses the term “nucleobase” which embraces naturally-occurring nucleotides as well as modifications of nucleotides that can be polymerized.

Methods of making polynucleotides of a predetermined sequence are well-known in the art. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

In various aspects, methods provided include use of polynucleotides which are DNA oligonucleotides, RNA oligonucleotides, or combinations of the two types. Modified forms of oligonucleotides are also contemplated which include those having at least one modified internucleotide linkage. Modified polynucleotides or oligonucleotides are described in detail herein below.

### II. Modified Polynucleotides

Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.” In specific embodiments, the competitor polynucleotide comprises phosphorothioate linkages.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still other embodiments, oligonucleotide mimetics wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., 1991, Science, 254: 1497-1500, the disclosures of which are herein incorporated by reference.

In still other embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligo consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where RH is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}CH₂—CH₂, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of oligonucleotides are described in detail in U.S. patent application NO. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples herein below.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In various aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226, the disclosures of which are incorporated by reference in their entireties herein. In various embodiments, the hairpin probe polynucleotide comprises a locked nucleic acid. In some embodiments, the hairpin probe polynucleotide comprises a plurality of locked nucleic acids.

Polynucleotides may also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-competitors such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

A “modified base” or other similar term refers to a composition which can pair with a natural base (e.g., adenine, guanine, cytosine, uracil, and/or thymine) and/or can pair with a non-naturally occurring base. In certain aspects, the modified base provides a Tₘ differential of 15, 12, 10, 8, 6, 4, or 2° C. or less. Exemplary modified bases are described in EP 1 072 679 and WO 97/12896.

By “nucleobase” is meant the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N′,N′-ethano-2,6-diaminopurine, 5-methylcytosine (mC), 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). The term “nucleosidic base” or “base unit” is further intended to include compounds such as heterocyclic compounds that can serve like nucleobases including certain “universal bases” that are not nucleosidic bases in the most classical sense but serve as nucleosidic bases. Especially mentioned as universal bases are 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

### III. Polynucleotide Structure—Length

In one aspect, a cleavable competitor polynucleotide has 10 nucleotides that are complementary to a target polynucleotide region. In various aspects, the cleavable competitor polynucleotide has at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, at least 50 nucleotides, at least 51 nucleotides, at least 52 nucleotides, at least 53 nucleotides, at least 54 nucleotides, at least 55 nucleotides, at least 56 nucleotides, at least 57 nucleotides, at least 58 nucleotides, at least 59 nucleotides, at least 60 nucleotides or more that are complementary to a target polynucleotide region.

In a related aspect, the PCR amplification primers each comprise at least 10 nucleotides in unique DNA sequence that are sufficiently complementary to second and third target polynucleotide regions as to allow hybridization between these complementary sequences under appropriate conditions. In various aspects, the PCR amplification primer polynucleotides comprise at least 11, at least 12, at least 13, at least 14, at least 15, at least 16, at least 17, at least 18, at least 19, at least 20, at least 21, at least 22, at least 23, at least 24 nucleotides, at least 25, at least about 30, at least about 35, at least about 40, at least about 45, at least about 50, at least about 60 or more nucleotides of a unique DNA sequence that is sufficiently complementary to the second and third target polynucleotide regions as to allow hybridization between the complementary sequences under appropriate conditions.

In some embodiments, the reverse primer polynucleotide is sufficiently complementary to a region of a polymerase-extended first polynucleotide so as to allow hybridization under appropriate conditions. In some embodiments, when the target polynucleotide is a double-stranded polynucleotide, the reverse primer is complementary to a complementary strand of the target polynucleotide. In some embodiments, the reverse primer is a combination of first and second polynucleotides, as defined herein.

In another embodiment, the hairpin probe polynucleotide comprises a first domain containing about 5 nucleotides, this first domain of the hairpin probe polynucleotide being complementary to a target DNA region C1 that is the cleavage product from a corresponding competitor. In various aspects, the second polynucleotide comprises a first domain containing at least 6, at least 7, at least 8, at least 9, at least 10, at least 11, at least 12, at least 13, at least 14, at least 15, at least 16, at least 17, at least 18, 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 or more nucleotides, the first domain of this hairpin probe polynucleotide being complementary, or sufficiently complementary, so as to recognize and bind to a C1 target DNA region that is derived from cleavage of its corresponding competitor polynucleotide.

In a related aspect, the second domain of the hairpin probe polynucleotide comprises 10 nucleotides of a unique DNA sequence that is sufficiently self-complementary so as to allow hairpin formation under appropriate conditions. In various aspects, the second domain of the second polynucleotide comprises at least 11, at least 12, at least 13, at least 14, at least 15, at least 16, at least 17, at least 18, at least 19, at least 20, at least 21, at least 22, at least 23, at least 24, at least 25, at least about 30, at least about 35, at least 40, at least about 45, at least about 50, at least about 60 or more nucleotides of a unique DNA sequence that is sufficiently self-complementary so as to allow hairpin formation between the two sufficiently complementary sequences under appropriate conditions.

In some embodiments, compositions and methods described herein include a second set of polynucleotides with the characteristics described above for competitor, primer and probe polynucleotides. In some embodiments, a plurality of sets is contemplated. These additional sets of competitor, primer and probe polynucleotides can have any of the characteristics described for competitor, primer and probe polynucleotides.

### IV. Polynucleotide Base Structure

In some embodiments, the competitor polynucleotide is comprised of DNA, modified DNA, RNA, modified RNA, PNA, or combinations thereof. In other embodiments, the primer and probe polynucleotides are comprised of DNA, modified DNA, RNA, modified RNA, PNA, or combinations thereof.

### V. Polynucleotide Structure—Blocking Groups

Blocking groups are incorporated as needed when polymerase extension from a 3′ region of a polynucleotide is undesirable. For example, the competitor and probe polynucleotides, in another aspect, further comprise a blocking group at the 3′ end to prevent extension by an enzyme that is capable of synthesizing a nucleic acid. Blocking groups useful in the practice of the methods include but are not limited to a 3′ phosphate group, a 3′ amino group, a dideoxy nucleotide, a six carbon glycol spacer (and in one aspect the six carbon glycol spacer is hexanediol) and inverted deoxythymidine (dT).

### VI. Hybridization Conditions

“Stringent conditions” as used herein can be determined empirically by the worker of ordinary skill in the art and will vary based on, e.g., the length of the primer, complementarity of the primer, concentration of the primer, the salt concentration (i.e., ionic strength) in the hybridization buffer, the temperature at which the hybridization is carried out, length of time that hybridization is carried out, and presence of factors that affect surface charge of the polynucleotides. In general, stringent conditions are those in which the polynucleotide is able to bind to its complementary sequence preferentially and with higher affinity relative to any other region on the target. Exemplary stringent conditions for hybridization to its complement of a polynucleotide sequence having 20 bases include without limitation about 50% G+C content, 50 mM salt (Na⁺), and an annealing temperature of 60° C. For a longer sequence, specific hybridization is achieved at higher temperature. In general, stringent conditions are such that annealing is carried out about 5° C. below the melting temperature of the polynucleotide. The “melting temperature” is the temperature at which 50% of polynucleotides are complementary to a target polynucleotide in equilibrium at definite ion strength, pH and polynucleotide concentration.

### VII. Methods of Use

### A. PCR

One of ordinary skill in the art will recognize that the polynucleotide primer combinations of the present invention can be used to prime either one or both ends of a given PCR amplicon. As used herein, an “amplicon” is understood to mean a portion of a polynucleotide that has been synthesized using amplification techniques. It is contemplated that any of the methods of the present invention that comprise more than one polynucleotide combination may utilize any combination of standard primer and polynucleotide combination, provided at least one of the primers is a polynucleotide combination as described herein.

In various embodiments, the target polynucleotide includes but is not limited to chromosomal DNA, genomic DNA, plasmid DNA, cDNA, RNA, a synthetic polynucleotide, a single stranded polynucleotide, or a double stranded polynucleotide.

### B. Multiplexing

In related embodiments, multiplex PCR is performed using at least two polynucleotide primers to amplify more than one polynucleotide product. In some aspects of these embodiments, each polynucleotide primer used for multiplex PCR is a polynucleotide combination as disclosed herein. In other aspects, at least one polynucleotide primer used for multiplex PCR is a polynucleotide combination as disclosed herein.

### C. Real-Time PCR

Primer combinations with cleavable competitors and hairpin probes are useful for real-time PCR. Analysis and quantification of rare transcripts, detection of limiting pathogens, diagnostics of rare cancer cells with mutations, or low levels of aberrant gene methylation in cancer patients are the problems that can be solved by improved real-time PCR assays that combine high sensitivity and specificity of target amplification, high specificity of target detection, the ability to selectively amplify and detect a small number of cancer-specific mutant alleles or abnormally methylated promoters in the presence of thousands of copies of normal DNA, analysis and quantification of low copy number RNA transcripts, detection of fluorescence traces the ability to multiplex 4-5 different targets in one assay to maximally utilize capabilities of current real-time thermal cyclers. A fluorophore is positioned at the 3′ end of the hairpin probe polynucleotide, and a quencher is positioned at the junction of the single-stranded and hairpin portions of the probe polynucleotide. In this arrangement, no fluorescence is detected when the self-complementary hairpin sequences are hybridized (since the fluorophore is positioned adjacent to the quencher). However, following extension of the C1 competitor cleavage product on the single stranded portion of the hairpin probe, distance between the fluorophore and the quencher occurs, resulting in a detectable fluorescent signal.

In some aspects, the above embodiments further comprise a reverse primer polynucleotide. The reverse primer is complementary to a region in the polynucleotide created by extension of the first polynucleotide. As is apparent, in some embodiments the reverse primer is also complementary to the complementary strand of the target polynucleotide when the target polynucleotide is one strand of a double-stranded polynucleotide. Inclusion of a reverse primer allows for amplification of the target polynucleotide. In various aspect, the reverse primer is a “simple” primer wherein the sequence of the reverse primer is designed to be sufficiently complementary over its entire length to hybridize to a target sequence over the entire length of the primer. A simple primer of this type is in one aspect, 100% complementary to a target sequence, however, it will be appreciated that a simple primer with complementarity of less than 100% is useful under certain circumstances and conditions.

In other aspects, a reverse primer is a separate polynucleotide primer combination that specifically binds to regions in a sequence produced by extension of a polynucleotide from the first domain of the first polynucleotide in a primer pair combination used in a first reaction.

In various aspects, the methods described herein provide a change in sequence detection from a sample with a non-target polynucleotide compared to sequence detection from a sample with a target polynucleotide. In some aspects, the change is an increase in detection of a target polynucleotide in a sample compared to sequence detection from a sample with a non-target polynucleotide. In some aspects, the change is a decrease in detection of a target polynucleotide in a sample compared to sequence detection from a sample with a non-target polynucleotide.

### VIII. Enzymes

In some aspects of any of the methods, the extension is performed by an enzyme that is capable of synthesizing a nucleic acid. The enzymes useful in the practice of the invention include but are not limited to a DNA polymerase (which can include a thermostable DNA polymerase, e.g., a Taq DNA polymerase), RNA polymerase, and reverse transcriptase. Non-limiting examples of enzymes that may be used to practice the present invention include but are not limited to Deep VentR™ DNA Polymerase, LongAmp™ Taq DNA Polymerase, Phusion™ High-Fidelity DNA Polymerase, Phusion™ Hot Start High-Fidelity DNA Polymerase, Kapa High-Fidelity DNA Polymerase, Q5 High-Fidelity DNA Polymerase, Platinum Pfx High-Fidelity Polymerase, Pfu High-Fidelity DNA Polymerase, Pfu Ultra High-Fidelity DNA Polymerase, KOD High-Fidelity DNA Polymerase, iProof High-Fidelity Polymerase, High-Fidelity 2 DNA Polymerase, Velocity High-Fidelity DNA Polymerase, ProofStart High-Fidelity DNA Polymerase, Tigo High-Fidelity DNA Polymerase, Accuzyme High-Fidelity DNA Polymerase, VentR® DNA Polymerase, DyNAzyme™ II Hot Start DNA Polymerase, Phire™ Hot Start DNA Polymerase, Phusion™ Hot Start High-Fidelity DNA Polymerase, Crimson LongAmp™ Taq DNA Polymerase, DyNAzyme™ EXT DNA Polymerase, LongAmp™ Taq DNA Polymerase, Phusion™ High-Fidelity DNA Polymerase, Taq DNA Polymerase with Standard Taq (Mg-free) Buffer, Taq DNA Polymerase with Standard Taq Buffer, Taq DNA Polymerase with ThermoPol II (Mg-free) Buffer, Taq DNA Polymerase with ThermoPol Buffer, Crimson Taq™ DNA Polymerase, Crimson Taq™ DNA Polymerase with (Mg-free) Buffer, Phire™ Hot Start DNA Polymerase, VentR® (exo-) DNA Polymerase, Hemo KlenTaq™, Deep VentR™ (exo-) DNA Polymerase, Deep VentR™ DNA Polymerase, DyNAzyme™ EXT DNA Polymerase, Hemo KlenTaq™, LongAmp™ Taq DNA Polymerase, ProtoScript® AMV First Strand cDNA Synthesis Kit, ProtoScript® M-MuLV First Strand cDNA Synthesis Kit, Bst DNA Polymerase, Full Length, Bst DNA Polymerase, Large Fragment, 9° Nm DNA Polymerase, DyNAzyme™ II Hot Start DNA Polymerase, Hemo KlenTaq™, Sulfolobus DNA Polymerase IV, Therminator™ γ DNA Polymerase, Therminator™ DNA Polymerase, Therminator™ II DNA Polymerase, Therminator™ III DNA Polymerase, Bsu DNA Polymerase, Large Fragment, DNA Polymerase I (E. coli), DNA Polymerase I, Large (Klenow) Fragment, Klenow Fragment (3′→5′ exo-), phi29 DNA Polymerase, T4 DNA Polymerase, T7 DNA Polymerase (unmodified), Terminal Transferase, Reverse Transcriptases and RNA Polymerases, E. coli Poly(A) Polymerase, AMV Reverse Transcriptase, M-MuLV Reverse Transcriptase, phi6 RNA Polymerase (RdRP), Poly(U) Polymerase, SP6 RNA Polymerase, and T7 RNA Polymerase.

### IX. Labels

In various aspects of the methods of the disclosure, detection of competitor cleavage by an enzyme is contemplated. In some aspects, the hairpin probe polynucleotide comprises a label. In some of these aspects the label is fluorescent. Methods of labeling oligonucleotides with fluorescent molecules and measuring fluorescence are well known in the art. Fluorescent labels useful in the practice of the invention include but are not limited to 1,8-ANS (1-Anilinonaphthalene-8-sulfonic acid), 1-Anilinonaphthalene-8-sulfonic acid (1,8-ANS), 5-(and-6)-Carboxy-2′, 7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), BO-PRO-1-DNA, BO-PRO-3-DNA, BOBO-1-DNA, BOBO-3-DNA, BODIPY 650/665-X, MeOH, BODIPY FL conjugate, BODIPY FL, MeOH, Bodipy R6G SE, BODIPY R6G, MeOH, BODIPY TMR-X antibody conjugate pH 7.2, Bodipy TMR-X conjugate, BODIPY TMR-X, MeOH, BODIPY TMR-X, SE, BODIPY TR-X phallacidin pH 7.0, BODIPY TR-X, MeOH, BODIPY TR-X, SE, BOPRO-1, BOPRO-3, Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, Cy 5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, DiI, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, Ethidium Bromide, Ethidium homodimer, Ethidium homodimer-1-DNA, eYFP (Enhanced Yellow Fluorescent Protein), FDA, FITC, FITC antibody conjugate pH 8.0, FlAsH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Ca, Fura-2 Ca2+, Fura-2, high Ca, Fura-2, no Ca, GFP (S65T), HcRed, Hoechst 33258, Hoechst 33258-DNA, Hoechst 33342, Indo-1 Ca2+, Indo-1, Ca free, Indo-1, Ca saturated, JC-1, JC-1 pH 8.2, Lissamine rhodamine, LOLO-1-DNA, Lucifer Yellow, CH, LysoSensor Blue, LysoSensor Blue pH 5.0, LysoSensor Green, LysoSensor Green pH 5.0, LysoSensor Yellow pH 3.0, LysoSensor Yellow pH 9.0, LysoTracker Blue, LysoTracker Green, LysoTracker Red, Magnesium Green, Magnesium Green Mg2+, Magnesium Orange, Marina Blue, mBanana, mCherry, mHoneydew, MitoTracker Green, MitoTracker Green FM, MeOH, MitoTracker Orange, MitoTracker Orange, MeOH, MitoTracker Red, MitoTracker Red, MeOH, mOrange, mPlum, mRFP, mStrawberry, mTangerine, NBD-X, NBD-X, MeOH, NeuroTrace 500/525, green fluorescent Nissl stain-RNA, Nile Blue, EtOH, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, PO-PRO-1, PO-PRO-1-DNA, PO-PRO-3, PO-PRO-3-DNA, POPO-1, POPO-1-DNA, POPO-3, Propidium Iodide, Propidium Iodide-DNA, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodaminen Green pH 7.0, Rhodol Green antibody conjugate pH 8.0, Sapphire, SBFI-Na+, Sodium Green Na+, Sulforhodamine 101, SYBR Green I, SYPRO Ruby, SYTO 13-DNA, SYTO 45-DNA, SYTOX Blue-DNA, Tetramethylrhodamine antibody conjugate pH 8.0, Tetramethylrhodamine dextran pH 7.0, Texas Red-X antibody conjugate pH 7.2, TO-PRO-1-DNA, TO-PRO-3-DNA, TOTO-1-DNA, TOTO-3-DNA, TRITC, X-Rhod-1 Ca2+, YO-PRO-1-DNA, YO-PRO-3-DNA, YOYO-1-DNA, and YOYO-3-DNA.

Other labels besides fluorescent molecules can be used, such as chemiluminescent molecules, which will give a detectable signal or a change in detectable signal upon hybridization, and radioactive molecules.

In some embodiments, the hairpin probe polynucleotide comprises a quencher that attenuates the fluorescence signal of a label. Quenchers contemplated for use in practice of the methods of the invention include but are not limited to Black Hole Quencher 1, Black Hole Quencher-2, Iowa Black FQ, Iowa Black RQ, Zen quencher, and Dabcyl. G-base.

### X. Modified Polynucleotide Combinations

Modified polynucleotides that are more sensitive to changes in template polynucleotide sequence than the basic polynucleotides can be used for development of more specific PCR-based diagnostic assays and for more sensitive PCR detection of rare DNA mutations in, e.g., cancer tissues.

The references cited herein throughout, to the extent that they provide exemplary procedural or other details supplementary to those set forth herein, are all specifically incorporated herein by reference.

### EXAMPLES

A person of skill in the art will appreciate that when primers or primer combinations are referred to as being in “forward” or “reverse” orientations, these designations are arbitrary conventions used in describing PCR reactions and the structural relationship of the primers and the template. Thus, as is apparent to a person of skill in the art, re-orienting a PCR schematic diagram by flipping it 180° would result in “forward” primers becoming “reverse” primers and “reverse” primers becoming “forward” primers, and as such, designation of, for example, one primer combination as a forward primer or a reverse primer is not a limitation on the structure or use of that particular primer combination.

### Example 1

### Sequence Specificity and Mismatch Discrimination of RNase H1 for RNA/DNA Hybrids

Materials: FIG. 27

Match DNA oligo on lanes 2, 4, 6 & 8 12-400 (Table 1)
Mismatch DNA oligo on lanes 1, 3, 5 & 7 12-439 (Table 1)
DNA/RNA oligo lane 1& 2 12-399
DNA/RNA oligo lane 3 & 4 12-448
DNA/RNA oligo lane 5 & 6 12-449
DNA/RNA oligo lane 7 & 8 12-450
Hybridase (Illumina Cat # H39500)
10x iTaq Buffer (Bio-Rad Cat # 170-8875)
50 mM Mg (Thermo Scientific Cat # F-510MG)
DNA resuspension buffer (Teknova Cat # T0227)

Method:

RNase H1 cleavage assay was performed in 25 ul reactions containing 10 pmol of RNA oligo, 15 pmol of DNA oligo, 1× iTaq buffer, 3mM Mg and DNA resuspension buffer. Cleavage assay was carried out in the presence of 5U of Hybridase at 95 C for 20 seconds followed by 65 C for 2 minutes. Samples were then immediately put on ice and re-suspended in formamide loading buffer. Samples were boiled for 2 minutes and run under denaturing conditions on a pre-cast 15% TBE-Urea polyacrylamide gel (Invitrogen, Cat #EC68852Box), stained with SYBR Gold stain (Invitrogen, Cat #S11494), visualized on a Dark Reader light box (Clare Chemical Research) and photographed using a digital camera.

Results:

As seen in FIG. 27 the match DNA oligo is depicted by M while the mismatch DNA oligo is depicted by MM. The DNA oligos are 34 bp long while the RNA oligos are 38 bp long. It has been shown earlier that RNase H1 cleaves the 4 RNA bases at the center. This results in cleavage of the rCrArUrG containing oligo in a match RNA/DNA hybrid (lane 1) in the middle (between second and third RNA bases) which gives 2 cleavage products which are both 19 bp in length. RNA containing oligo in a mismatch RNA/DNA hybrid (lane 2) which has a rA:C mismatch does not cleave as efficiently. Similarly rGrCrArU containing oligo in a match hybrid (lane 3) gets cleaved to produce 2 products of 18bp and 20 bp length (can't be easily separated on gel) while the rA:C mismatch (lane 4) is not cleaved as efficiently. rUrGrCrA sequence is not cleaved at all by RNase H1 under these reaction conditions whether it is present in a match (lane 5) or a mismatch RNA/DNA hybrid (lane 6). rArUrGrA oligo is cleaved in a match (lane 7) and gives 2 products which are 18 bp and 20 bp in length. The mismatch rA:C does not get cleaved at all by RNase H1 (lane 8). 17 bp, 19 bp and 21 bp oligos are shown as reference markers.

Conclusions:

Analysis of the cleavage products in FIG. 27 indicates that RNase H1 cleaved some RNA sequences in an RNA/DNA hybrid more efficiently than others. For example the order of preference for sequences in this case would be CAUG>GCAU>AUGA>UGCA. Moreover it was also observed that RNase H1 cleaves match RNA sequences in a RNA/DNA hybrid (rA on T) much more efficiently than mismatch RNA sequences (rA on C). Thus, RNase H1 mediated cleavage is both sequence specific and mismatch sensitive.

### Example 2

### RNase H1 Kinetics for an Efficiently Cleavedversus an Inefficiently Cleavedrna/DNA Hybrid Sequence

Materials: FIG. 28

Trackit DNA marker (Life Technologies Cat # 10488-022)
Match DNA oligo on lanes 2, 3, 4, 5 & 6 12-400 (Table 1)
Match DNA oligo on lanes 7, 8, 9, 10 & 11 12-397 (Table 1)
DNA/RNA oligo on lanes 2, 3, 4, 5 & 6 12-399 (Table 1)
DNA/RNA oligo on lanes 7, 8, 9, 10 & 11 12-284 (Table 1)
Hybridase (Illumina Cat # H39500)
10x iTaq Buffer (Bio-Rad Cat # 170-8875)
50 mM Mg (Thermo Scientific Cat # F-510MG)
DNA resuspension buffer (Teknova Cat # T0227)

Method:

RNase H1 cleavage assay was performed in 25 ul reactions containing 10 pmol of RNA oligo, 15 pmol of DNA oligo, lx iTaq buffer, 3 mM Mg and DNA resuspension buffer. Cleavage assay was carried out in the presence of 5U of Hybridase at 95 C for 20 seconds followed by 65 C for either 0 secs, 30 secs, 1 minute, 5 minute and 10 minutes. Samples were then immediately put on ice and re-suspended in formamide loading buffer. Samples were boiled for 2 minutes and run on a pre-cast 15% TBE-Urea polyacrylamide gel (Invitrogen, Cat #EC68852Box), stained with SYBR Gold stain (Invitrogen, Cat #S11494), visualized on a Dark Reader light box (Clare Chemical Research) and photographed using a digital camera.

Results:

As shown in FIG. 28 the DNA oligos are 34 bp long while the RNA oligos are 38 bp long. rCrArUrG containing oligo in a match RNA/DNA hybrid (lane 2-6) is fully cleaved at 30 sec incubation (lane2) while rArUrGrC containing oligo in a match RNA/DNA hybrid (lane 7-11) takes about 5 minutes to cleave partially (lane 10) and 10 minutes to cleave significantly (lane 11). 19 bp oligo is used as a marker.

Conclusions:

Analysis of the gel data in FIG. 28 reveals that enzyme kinetics for cleavage is much faster for RNA/DNA hybrid sequences which are efficient substrates for RNase H1 and much slower for RNA/DNA hybrid sequences which are inefficient substrates for RNase H1.

### Example 3

### Competitor Mediated Inhibition of the Wild-Type Signal Using Forward Primers Which are Either Overlapping or Non-Overlapping with the Competitor

Materials: (FIG. 29)

Forward primer in FIG. 29 A depicted with black or triangular line 12-446
(Table 1)
Reverse primer in FIG. 29 A depicted with black or triangular line 11-63
(Table 1)
Forward primer in FIG. 29 B depicted with black or triangular line 12-478
(Table 1)
RNA bases containing competitor oligo in FIG. 29 12-514 (Table 1)
Phusion High-Fidelity DNA polymerase (Thermo Scientific Cat # F-530L)
100 mM dNTP Set (Life Technologies Cat # 10297-018)
SYTO ® 9 (Life Technologies Cat # S-34854)
DNA resuspension buffer (Teknova Cat # T0227)
Glycerol (VWR Cat # 56-81-5)
5x HF Phusion Buffer (Thermo Scientific cat # F-518)
Wild Type EGFR DNA template containing plasmid (Genscript)
Bio-Rad CFX-96 thermocycler

Method:

PCR was set-up in 25 ul reactions using 200 nM of forward and reverse primers, 1600 nM of competitor, 1× Phusion HF Buffer, 200 uM of dNTP, 0.5 U of Phusion high fidelity DNA polymerase, 4% Glycerol, 1.6 uM of SYTO® 9, DNA resuspension buffer and 1000 copies of wild-type EFGR template containing plasmid. Cycling conditions were as follows: 1. 95 C for 3 minutes, 2. 95 C for 10 seconds, 3. 75 C for 15 seconds, 4. 65 C for 1 minute, Go-to 2 repeat 6 cycles, 5. 90 C for 10 seconds, 6. 75 C for 15 seconds, 7. 65 C for 1 minute, Go to 5 repeat 54 cycles. PCR products were detected with SYTO® 9 dye under the SYBR/FAM filter in Bio-Rad CFX-96 thermocycler.

Results:

As seen in FIG. 29 A. when a locus specific PCR is performed with just the forward primer (overlapping with the competitor) and the reverse primer to detect 1000 copies of wild-type EGFR (black line graph), the average Ct is 27.9. When an RNA containing competitor is used (black triangular line graph), it inhibits the locus specific detection of wild-type EGFR leading to a Ct difference of 12.03. However as seen in FIG. 29 B, when a locus specific PCR is performed with just the forward primer (non-overlapping with the competitor) and the reverse primer to detect 1000 copies of wild-type EGFR (black line graph), the average Ct is 27.77. When an RNA containing competitor is used (black triangular line graph), it inhibits the locus specific detection of wild-type EGFR leading to a Ct difference of 4.43.

Conclusion:

The PCR assay in FIG. 29 was carried out using Phusion which is a HiFi polymerase lacking strand displacement activity. This will prevent the forward primer from displacing the competitor during extension. Even in this scenario the inhibition of an overlapping forward primer is much more efficient than a non-overlapping primer which suggests that an overlapping primer and competitor combination would be much better in inhibiting the wild-type DNA signal as compare to a non-overlapping primer and competitor.

### Example 4

### Mismatch Discrimination Between Wild-Type and Mutant Signals Using RNase H1 in a PCR Assay

Materials: FIG. 30

Forward primer in FIG. 30 12-446 (Table 1)
Reverse primer in FIG. 30 11-63 (Table 1)
RNA/DNA competitor oligo in FIG. 30 B 12-441 (Table 1)
Taq Man probe oligo in FIG. 26 11-64 (Table 1)
iQ Supermix (Bio-Rad Cat # 170-8864)
Glycerol (VWR Cat # 56-81-5)
Hybridase (Illumina Cat # H39500
DNA resuspension buffer (Teknova Cat # T0227)
Wild-type EGFR template containing plasmid (Genscript)
Mutant EGFR T790 M template containing plasmid (Genscript)
Bio-Rad CFX-96 thermocycler

Methods:

PCR was set-up in 25 ul reactions using 100 nM of forward and reverse primers, 240 nM of Taq-Man probe, 800 nM of competitor, 1× iQ supermix, DNA resuspension buffer, 10U Hybridase or 4% Glycerol and 1000 copies of either wild-type EGFR template containing plasmid or mutant EGFR T790M template containing plasmid. Cycling conditions were as follows: 1. 95 C for 3 minutes, 2. 95 C for 10 seconds, 3. 75 C for 15 seconds, 4. 65 C for 1 minute, Go-to 2 repeat 6 cycles, 5. 90 C for 10 seconds, 6. 75 C for 15 seconds, 7. 65 C for 1 minute, Go to 5 repeat 54 cycles. PCR products were detected under the SYBR/FAM filter in Bio-Rad's CFX-96 thermocycler.

Results:

As seen in FIG. 30 A locus specific PCR was used to detect 1000 copies of EGFR wild-type plasmid and 1000 copies of EGFR T790M plasmid. The Ct values for both were very close with the average wild-type Ct coming up at 21.11 and the average mutant Ct coming up at 21.16. As seen in FIG. 30 B when competitor and RNase H1 were added to this assay the wild-type EGFR signal was inhibited much more significantly (average Ct of 34.76) than the mutant T790M EGFR signal (average Ct of 23.74). The Ct difference between wild-type and mutant signal was 11.02 which translates into a difference of about 2000 fold.

Conclusion:

The PCR assay results depicted in FIG. 30 indicate that RNase H1 can distinguish between a mutant and a wild-type signal even in a PCR assay. The discrimination of the wild-type signal leads to an amplification fold difference of 2000 between mutant and wild-type.

TABLE 1

| | | SEQ |
|---|---|---|
| | | ID |
| Primer | Sequence | NO |
| 12-400 | CACCGTGCAGCTCATCATGCAGCTCATGCCCTTC | 1 |
| 12-439 | CACCGTGCAGCTCATCACGCAGCTCATGCCCTTC | 2 |
| 12-399 | CCGAAGGGCATGAGCTG-rCrArUrG-ATGAGCTGCACGGTGGA/3Phos/ | 3 |
| 12-448 | CCGAAGGGCATGAGCT-rGrCrArU-GATGAGCTGCACGGTGGA/3Phos/ | 4 |
| 12-449 | CCGAAGGGCATGAGC-rUrGrCrA-TGATGAGCTGCACGGTGGA/3Phos/ | 5 |
| 12-450 | CCGAAGGGCATGAGCTGC-rArUrGrA-TGAGCTGCACGGTGGA/3Phos/ | 6 |
| 12-397 | CGAAGGGCATGAGCTGCATGATGAGCTGCACGGT | 7 |
| 12-284 | CCACCGTGCAGCTCATCrArUrGrCAGCTCATGCCCTTCGGC/3Phos/ | 8 |
| 12-446 | GGCAACCGAAGGGCATGAGCT | 9 |
| 11-63 | ATGCGAAGCCACACTGACGT | 10 |
| 12-478 | AGTCCAGGAGGCAGCCGA | 11 |
| 12-514 | AGGGCATGAGCTGrCrArUrGATGAGCTGCAC*G*G*T/3Phos/ | 12 |
| 12-441 | AGGGCATGAGCTG-rCrArUrG-ATGAGCTGCACGGT/3Phos/ | 13 |
| 11-64 | /56-FAM/TACGTGATG/ZEN/GCCAGCGTGGAC/3IABkFQ/ | 14 |

What is claimed is:

1. A composition comprising a first polynucleotide and a second polynucleotide, wherein: (A) the first polynucleotide comprises a sequence such that: (i) the first polynucleotide has a fully complementary domain to a target polynucleotide (T1) such that the first polynucleotide is able to hybridize to T1 under appropriate conditions, and the sequence comprises a RNA base that is susceptible to cleavage by a ribonuclease when the RNA base is hybridized to T1; and (ii) the first polynucleotide is mismatched to a non-target polynucleotide region (T1*) at the position of the RNA base or 1, 2 or 3 nucleotides adjacent to the RNA base; and (iii) T1* is a sequence variant of T1; and (B) the second polynucleotide comprises a sequence such that: (iv) the second polynucleotide is fully complementary to a target polynucleotide region (T2) and a non-target polynucleotide region (T2) that overlaps T1 and T1* by at least one nucleotide, wherein T2 is upstream of T1 and T1*.

2. The composition of claim 1, wherein the RNA base on the first polynucleotide is located at the midpoint of the first polynucleotide.

3. The composition of claim 1 or claim 2, further comprising at least one additional RNA base on the first polynucleotide located immediately adjacent to the first RNA base.

4. The composition of claim 3 wherein the first polynucleotide comprises at least 4 consecutive RNA bases.

5. The composition of claim 4 wherein one or more RNA bases are susceptible to cleavage by a ribonuclease when the polynucleotide is hybridized to the target sequence T1.

6. The composition of claim 5 wherein modified nucleotides at one or more RNA bases renders the one or more bases resistant to cleavage by a ribonuclease.

7. The composition of claims 1-6 wherein the 3′ terminus of the first polynucleotide is blocked from initiation of extension by a DNA polymerase.

8. The composition of any one of claims 1-7 wherein the first polynucleotide comprises a detectable marker and a moiety that quenches the detectable marker.

9. The composition of claim 8 wherein the detectable marker and the moiety are on opposite sides of the RNA base, and in a configuration that prevents detection of the detectable marker.

10. The composition of claim 9 wherein cleavage of the first polynucleotide results in detection of the detectable marker.

11. The composition of any one of claims 1-10 wherein T2 overlaps T1 and T1* by at least about 1 to at least about 50 nucleotides.

12. The composition of any one of claims 1-11 wherein the ribonuclease includes but is not limited to RNase H2 or RNase H1.

13. A method of initiating polymerase extension on a target polynucleotide in a sample using the composition of any one of claims 1-12; wherein the sample comprises a target polynucleotide that comprises (i) a sequence T1 in a first region that is fully complementary to the sequence of a domain in the first polynucleotide; and (ii) a sequence T2 that is fully complementary to the sequence in the second polynucleotide; the method comprising the step of (a) contacting the sample with the composition and a polymerase under conditions that allow extension of a sequence from T2 following cleavage and dissociation of the first polynucleotide.

14. A method of amplifying a target polynucleotide in a sample using the composition of any one of claims 1-12, wherein: the sample comprises a mixture of (i) a target polynucleotide having a sequence in a first region (T1) that is fully complementary to the sequence of a domain in the first polynucleotide, and a sequence in a second region (T2) that is fully complementary to the sequence in the second polynucleotide; and (ii) one or more non-target polynucleotides that are not fully complementary to T1; the method comprising the steps of: (a) contacting the sample with the composition and a polymerase under conditions that allow extension of a sequence (S) from T2, wherein the sequence is complementary to the target polynucleotide when the target polynucleotide is present in the sample; (b) denaturing the sequence (S) extended from T2 from the target polynucleotide, and (c) repeating step (a) in the presence of a third polynucleotide having a sequence complementary to a region (T3) in the sequence extended from T2 in step (b) to amplify the target polynucleotide, wherein extension and amplification of the target polynucleotide to generate a product occurs when the first polynucleotide is fully complementary to the sequence in T1, but is less efficient or does not occur when the first polynucleotide is not fully complementary to the sequence in a non-target sequence T1*; and wherein steps (a)-(c) are followed by further extension and amplification of the product when the first polynucleotide hybridizes to T1 or the second polynucleotide hybridizes to T2, and the third polynucleotide hybridizes to T3 in the presence of the polymerase.

15. The method of claim 14, further comprising: (iii) a fourth polynucleotide having a sequence that is fully complementary to a region T4 in a second target polynucleotide in the sample, such that the fourth polynucleotide is able to hybridize to T4 under appropriate conditions, and the sequence comprises a RNA base that is susceptible to cleavage by a ribonuclease when the RNA base is hybridized to T4; and (iv) a fifth polynucleotide having a sequence that is fully complementary to a region T5 in a second target polynucleotide in the sample, wherein T5 overlaps T4 and T4* by at least one nucleotide, and wherein T5 is upstream of T4 and T4*; wherein (v) the fourth polynucleotide is mismatched to a non-target polynucleotide region (T4*) at the position of the RNA base or 1, 2 or 3 bases adjacent to the RNA base; and wherein the sample comprises a mixture of (i) a target polynucleotide having a sequence in a first region (T4) that is fully complementary to the sequence of a domain in the fourth polynucleotide, and a sequence in a second region (T5) that is fully complementary to the sequence in the fifth polynucleotide; and (ii) one or more non-target polynucleotides that are not fully complementary to T4.

16. The method of any one of claims 13-15 further comprising the step of adding a ribonuclease at step (a).


SEQUENCE LISTING (14 sequences)

SEQ ID NO: 1 - DNA, 34 bp, Artificial Sequence
  Synthetic oligonucleotide
caccgtgcagctcatcatgcagctcatgcccttc

SEQ ID NO: 2 - DNA, 34 bp, Artificial Sequence
  Synthetic oligonucleotide
caccgtgcagctcatcacgcagctcatgcccttc

SEQ ID NO: 3 - DNA, 38 bp, Artificial Sequence
  Synthetic oligonucleotide
  misc_feature (18)..(18): Ribo base
  misc_feature (19)..(19): Ribo base
  misc_feature (20)..(20): Ribo base
  misc_feature (21)..(21): Ribo base
  misc_feature (38)..(38): 3′ phosphate
ccgaagggcatgagctgcaugatgagctgcacggtgga

SEQ ID NO: 4 - DNA, 38 bp, Artificial Sequence; identical to SEQ ID NO: 3
  Synthetic oligonucleotide
  misc_feature (17)..(17): Ribo base
  misc_feature (18)..(18): Ribo base
  misc_feature (19)..(19): Ribo base
  misc_feature (20)..(20): Ribo base
  misc_feature (38)..(38): 3′ phosphate

SEQ ID NO: 5 - DNA, 38 bp, Artificial Sequence
  Synthetic oligonucleotide
  misc_feature (16)..(16): Ribo base
  misc_feature (17)..(17): Ribo base
  misc_feature (18)..(18): Ribo base
  misc_feature (19)..(19): Ribo base
  misc_feature (38)..(38): 3′ phosphate
ccgaagggcatgagcugcatgatgagctgcacggtgga

SEQ ID NO: 6 - DNA, 38 bp, Artificial Sequence; identical to SEQ ID NO: 3
  Synthetic oligonucleotide
  misc_feature (19)..(19): Ribo base
  misc_feature (20)..(20): Ribo base
  misc_feature (21)..(21): Ribo base
  misc_feature (22)..(22): Ribo base
  misc_feature (38)..(38): 3′ phosphate

SEQ ID NO: 7 - DNA, 34 bp, Artificial Sequence
  Synthetic oligonucleotide
cgaagggcatgagctgcatgatgagctgcacggt

SEQ ID NO: 8 - DNA, 38 bp, Artificial Sequence
  Synthetic oligonucleotide
  misc_feature (18)..(18): Ribo base
  misc_feature (19)..(19): Ribo base
  misc_feature (20)..(20): Ribo base
  misc_feature (21)..(21): Ribo base
  misc_feature (38)..(38): 3′ phosphate
ccaccgtgcagctcatcaugcagctcatgcccttcggc

SEQ ID NO: 9 - DNA, 21 bp, Artificial Sequence
  Synthetic oligonucleotide
ggcaaccgaagggcatgagct

SEQ ID NO: 10 - DNA, 20 bp, Artificial Sequence
  Synthetic oligonucleotide
atgcgaagccacactgacgt

SEQ ID NO: 11 - DNA, 18 bp, Artificial Sequence
  Synthetic oligonucleotide
agtccaggaggcagccga

SEQ ID NO: 12 - DNA, 31 bp, Artificial Sequence
  Synthetic oligonucleotide
  misc_feature (14)..(14): Ribo base
  misc_feature (15)..(15): Ribo base
  misc_feature (16)..(16): Ribo base
  misc_feature (17)..(17): Ribo base
  misc_feature (28)..(29): Phosphorothioate linkage
  misc_feature (29)..(30): Phosphorothioate linkage
  misc_feature (30)..(31): Phosphorothioate linkage
  misc_feature (31)..(31): 3′ phosphate
agggcatgagctgcaugatgagctgcacggt

SEQ ID NO: 13 - DNA, 31 bp, Artificial Sequence; identical to SEQ ID NO: 12
  Synthetic oligonucleotide
  misc_feature (14)..(14): Ribo base
  misc_feature (15)..(15): Ribo base
  misc_feature (16)..(16): Ribo base
  misc_feature (17)..(17): Ribo base
  misc_feature (31)..(31): 3′ phosphate

SEQ ID NO: 14 - DNA, 21 bp, Artificial Sequence
  Synthetic oligonucleotide
  misc_feature (1)..(1): 5′ FAM fluorophore
  misc_feature (9)..(10): IDT internal Zen quencher
  misc_feature (21)..(21): 3′ IDT Iowa black quencher
tacgtgatggccagcgtggac